SEQUENCE LISTING (16 sequences)

SEQ ID NO: 1 - DNA, 3204 bp, Artificial Sequence
  cDNA sequence encoding HIV antigens Gag-RT-Nef (“GRN”) from Clade B
atgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggccggagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 2 - protein, 1067 aa, Artificial Sequence
  Amino acid sequence encoding HIV antigens Gag-RT-Nef (“GRN”) from Clade B
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 3 - DNA, 4665 bp, Artificial Sequence
  cDNA sequence encoding HIV antigens Gag-RT-integrase-Nef (“GRIN”) from Clade A
atggccgccagagccagcatcctgagcgggggcaagctggacgcctgggagaagatcagactgaggcctggcggcaagaagaagtaccggctgaagcacctggtgtgggccagcagagagctggatcgcttcgccctgaatcctagcctgctggagaccaccgagggctgccagcagatcatgaaccagctgcagcccgccgtgaaaaccggcaccgaggagatcaagagcctgttcaacaccgtggccaccctgtactgcgtgcaccagcggatcgacgtgaaggataccaaggaggccctggacaagatcgaggagatccagaacaagagcaagcagaaaacccagcaggccgctgccgacaccggcgacagcagcaaagtgagccagaactaccccatcatccagaatgcccagggccagatgatccaccagaacctgagccccagaaccctgaatgcctgggtgaaagtgatcgaggaaaaggccttcagccccgaagtgatccctatgttcagcgccctgagcgagggcgccaccccccaggacctgaacgtgatgctgaacattgtgggcggacaccaggccgccatgcagatgctgaaggacaccatcaatgaggaggccgccgagtgggacagactgcaccccgtgcaggccggacccatcccccctggccagatcagagagcccagaggcagcgacatcgccggcaccacctccacccctcaagaacagctgcagtggatgaccggcaaccctcccatccctgtgggcaacatctacaagcggtggatcatcctgggcctgaacaagattgtgcggatgtacagccccgtgtccatcctggatatcaagcagggccccaaggagcccttcagagactacgtggaccggttcttcaaggccctgagagccgagcaggccacccaggacgtgaagggctggatgaccgagaccctgctggtgcagaacgccaaccccgactgcaagagcatcctgaaggccctgggcagcggcgccacactggaggagatgatgaccgcctgccagggagtgggcggacccggccacaaggccagagtgctggccgaggccatgagccaggcccagcagaccaacatcatgatgcagcggggcaacttcagaggccagaagcggatcaagtgcttcaactgcggcaaggagggccacctggccagaaactgcagagcccccaggaagaagggctgctggaagtgtggcaaggaagggcaccagatgaaggactgcaccgagaggcaggccaatttcctgggcaagatttggcctagcagcaagggcagacccggcaatttcccccagagcagacccgagcccaccgcccctcccgccgagctgttcggcatgggcgagggcatcgccagcctgcccaagcaggagcagaaggacagagagcaggtgccccccctggtgtccctgaagtccctgttcggcaacgatcctctgagccagggatcccccatcagccccatcgagaccgtgcccgtgaccctgaagcccggcatggatggccccaaagtgaaacagtggcccctgaccgaggagaagattaaggccctgaccgaaatctgtaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagtccgtgacagtgctggatgtgggcgacgcctacttcagcgtgcccctggacgagaacttcaggaagtacaccgccttcaccatccccagcaccaacaacgagacccccggagtgagataccagtacaacgtgctgcctcagggctggaagggcagccccgccatcttccagagcagcatgaccaagatcctggagcccttccggagcaagaaccccgagatcatcatctaccagtacatggccgccctgtatgtgggcagcgatctggagatcggccagcacaggaccaagatcgaagagctgagggcccacctgctgagctggggcttcaccacccccgataagaagcaccagaaggagccccctttcctgtggatgggctacgagctgcaccccgataagtggaccgtgcagcccatcatgctgcccgataaggagagctggaccgtgaacgacatccagaaactggtgggcaagctgaattgggccagccaaatctacgccggcattaaagtgaagcagctgtgcaggctgctgagaggcgccaaagccctgacagacatcgtgacactgacagaggaggccgagctggagctggccgagaacagggagatcctgaaggaccccgtgcacggcgtgtactacgaccccagcaaggacctggtggccgagattcagaagcagggccaggaccagtggacctaccaaatctaccaggagcctttcaagaacctgaaaaccgggaagtacgccaggaagagaagcgcccacaccaacgatgtgaggcagctggccgaagtggtgcagaaagtggctatggagagcatcgtgatctggggcaagacccccaagttcaagctgcccatccagaaggagacctgggaaacctggtggatggactactggcaggccacctggattcctgagtgggagttcgtgaacaccccccctctggtgaagctgtggtatcagctggagaaggaccccatcctgggcgccgagaccttctacgtggacggagccgccaatagagagaccaagctgggcaaggccggctacgtgaccgacagaggcagacagaaagtggtgtctctgaccgagacaaccaaccagaaaaccgagctgcacgccatcctgctggccctgcaggacagcggcagcgaagtgaacatcgtgaccgactcccagtacgccctgggcatcattcaggcccagcccgatagaagcgagagcgagctggtgaaccagatcatcgagaagctgatcggcaaggacaaaatctacctgagctgggtgcccgcccacaagggcatcggcggcaacgagcaggtggacaagctggtgtccagcggcatccggaaagtgctgtttctggacggcatcgacaaggcccaggaggaccacgagagataccacagcaactggcggacaatggccagcgacttcaacctgcctcccatcgtggccaaggagatcgtggccagctgcgataagtgtcagctgaagggcgaggccatgcacggccaggtggactgcagccctggcatctggcagctggcctgcacccacctggagggcaaagtgattctggtggccgtgcacgtggccagcggctacatcgaggccgaagtgattcccgccgagaccggccaggagaccgcctacttcctgctgaagctggccggcagatggcccgtgaaagtggtgcacaccgccaacggcagcaacttcacctctgccgccgtgaaggccgcctgttggtgggccaatatccagcaggagttcggcatcccctacaaccctcagagccagggcgtggtggccagcatgaacaaggagctgaagaagatcatcggccaggtgagggaccaggccgagcacctgaaaacagccgtgcagatggccgtgttcatccacaacttcaagcggaagggcggcattggcggctacagcgccggagagcggatcatcgacatcatcgccaccgatatccagaccaaggaactgcagaagcagatcaccaagattcagaacttcagagtgtactaccgggacagcagggaccccatctggaagggccctgccaagctgctgtggaagggcgaaggcgccgtggtgatccaggacaacagcgacatcaaagtggtgccccggaggaaggccaagattctgcgggactacggcaaacagatggccggcgatgactgcgtggccggcaggcaggatgaggacagatctatgggcggcaagtggtccaagggcagcattgtgggctggcccgagatccgggagagaatgagaagagcccctgccgccgctcctggagtgggcgccgtgtctcaggatctggataagcacggcgccatcaccagcagcaacatcaacaaccccagctgtgtgtggctggaggcccaggaagaggaggaagtgggcttccctgtgagaccccaggtgcccctgagacccatgacctacaagggcgccttcgacctgagccacttcctgaaggagaagggcggcctggacggcctgatctacagccggaagcggcaggagatcctggatctgtgggtgtaccacacccagggctacttccccgactggcagaattacacccctggccctggagtgcggtatcccctgaccttcggctggtgcttcaagctggtgcctatggagcccgacgaagtggagaaggccacagagggcgagaacaacagcctgctgcaccctatctgccagcacggcatggacgatgaggagcgggaagtgctgatctggaagttcgacagcaggctggccctgaagcacagagcccaggaactgcacccagagttctacaaggactgctga

SEQ ID NO: 4 - protein, 1554 aa, Artificial Sequence
  Amino acid sequence encoding HIV antigens Gag-RT-integrase-Nef (“GRIN”) from Clade A
MAARASILSGGKLDAWEKIRLRPGGKKKYRLKHLVWASRELDRFALNPSLLETTEGCQQIMNQLQPAVKTGTEEIKSLFNTVATLYCVHQRIDVKDTKEALDKIEEIQNKSKQKTQQAAADTGDSSKVSQNYPIIQNAQGQMIHQNLSPRTLNAWVKVIEEKAFSPEVIPMFSALSEGATPQDLNVMLNIVGGHQAAMQMLKDTINEEAAEWDRLHPVQAGPIPPGQIREPRGSDIAGTTSTPQEQLQWMTGNPPIPVGNIYKRWIILGLNKIVRMYSPVSILDIKQGPKEPFRDYVDRFFKALRAEQATQDVKGWMTETLLVQNANPDCKSILKALGSGATLEEMMTACQGVGGPGHKARVLAEAMSQAQQTNIMMQRGNFRGQKRIKCFNCGKEGHLARNCRAPRKKGCWKCGKEGHQMKDCTERQANFLGKIWPSSKGRPGNFPQSRPEPTAPPAELFGMGEGIASLPKQEQKDREQVPPLVSLKSLFGNDPLSQGSPISPIETVPVTLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDENFRKYTAFTIPSTNNETPGVRYQYNVLPQGWKGSPAIFQSSMTKILEPFRSKNPEIIIYQYMAALYVGSDLEIGQHRTKIEELRAHLLSWGFTTPDKKHQKEPPFLWMGYELHPDKWTVQPIMLPDKESWTVNDIQKLVGKLNWASQIYAGIKVKQLCRLLRGAKALTDIVTLTEEAELELAENREILKDPVHGVYYDPSKDLVAEIQKQGQDQWTYQIYQEPFKNLKTGKYARKRSAHTNDVRQLAEVVQKVAMESIVIWGKTPKFKLPIQKETWETWWMDYWQATWIPEWEFVNTPPLVKLWYQLEKDPILGAETFYVDGAANRETKLGKAGYVTDRGRQKVVSLTETTNQKTELHAILLALQDSGSEVNIVTDSQYALGIIQAQPDRSESELVNQIIEKLIGKDKIYLSWVPAHKGIGGNEQVDKLVSSGIRKVLFLDGIDKAQEDHERYHSNWRTMASDFNLPPIVAKEIVASCDKCQLKGEAMHGQVDCSPGIWQLACTHLEGKVILVAVHVASGYIEAEVIPAETGQETAYFLLKLAGRWPVKVVHTANGSNFTSAAVKAACWWANIQQEFGIPYNPQSQGVVASMNKELKKIIGQVRDQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKILRDYGKQMAGDDCVAGRQDEDRSMGGKWSKGSIVGWPEIRERMRRAPAAAPGVGAVSQDLDKHGAITSSNINNPSCVWLEAQEEEEVGFPVRPQVPLRPMTYKGAFDLSHFLKEKGGLDGLIYSRKRQEILDLWVYHTQGYFPDWQNYTPGPGVRYPLTFGWCFKLVPMEPDEVEKATEGENNSLLHPICQHGMDDEEREVLIWKFDSRLALKHRAQELHPEFYKDC

SEQ ID NO: 5 - DNA, 2025 bp, Artificial Sequence
  cDNA sequence encoding gp140 from HIV Clade A
atgagggtgatggagatccagcggaactgccagcacctgctgagatggggcatcatgatcctgggcatgattatcatctgcagcaccgccgacaacctgtgggtgaccgtgtactacggcgtgcctgtgtggagagatgccgagaccaccctgttctgcgccagcgacgccaaggcctacagcaccgagaagcacaatgtgtgggccacccacgcctgcgtgcctaccgatcccaaccctcaggagatccccctggacaacgtgaccgaggagttcaacatgtggaagaacaacatggtggaccagatgcacgaggacatcatcagcctgtgggaccagagcctgaagccctgcgtgcagctgacccccctgtgcgtgaccctgaactgcagcaacgccagagtgaacgccaccttcaactccaccgaggacagggagggcatgaagaactgcagcttcaacatgaccaccgagctgcgggataagaagcagcaggtgtacagcctgttctaccggctggacatcgagaagatcaacagcagcaacaacaacagcgagtaccggctggtgaactgcaataccagcgccatcacccaggcctgccctaaggtgaccttcgagcccatccccatccactactgcgcccctgccggcttcgccatcctgaagtgcaacgacaccgagttcaatggcaccggcccctgcaagaatgtgagcaccgtgcagtgcacccacggcatcaagcccgtggtgtccacccagctgctgctgaacggcagcctggccgagagagaagtgcggatcaggagcgagaacatcgccaacaacgccaagaacatcatcgtgcagttcgccagccccgtgaagatcaactgcatccggcccaacaacaatacccggaagagctacagaatcggccctggccagaccttctacgccaccgacattgtgggcgacatcagacaggcccactgcaacgtgtccaggaccgactggaacaacaccctgagactggtggccaaccagctgcggaagtacttcagcaacaagaccatcatcttcaccaacagcagcggcggagacctggagatcaccacccacagcttcaattgtggcggcgagttcttctactgcaacacctccggcctgttcaatagcacctggaccaccaacaacatgcaggagtccaacgacaccagcaacggcaccatcaccctgccctgccggatcaagcagatcatccggatgtggcagcgcgtgggccaggccatgtacgcccctcccatcgagggcgtgattcgctgcgagagcaacatcaccggcctgatcctgaccagagatggcggcaacaacaattccgccaacgagaccttcagacctggcggcggagatatccgggacaactggcggagcgagctgtacaagtacaaggtggtgaagatcgagcccctgggcgtggcccccaccagagccaagagaagagtggtggagcgggagaagagagccgtgggcatcggcgccgtgtttctgggcttcctgggagccgccggatctacaatgggagccgccagcatcaccctgaccgtgcaggccagacagctgctgagcggcatcgtgcagcagcagagcaatctgctgagagccatcgaggcccagcagcagctgctgaagctgacagtgtggggcatcaagcagctgcaggccagggtgctggccgtggagagatacctgagggaccagcagctcctgggcatctggggctgcagcggcaagctgatctgcaccaccaacgtgccctggaatagcagctggagcaacaagagctacgacgacatctggcagaacatgacctggctgcagtgggacaaggagatcagcaactacaccgacatcatctacagcctgatcgaggagagccagaaccagcaggagaagaacgagcaggatctgctggccctggacaagtgggccaacctgtggaactggttcgacatcagcaagtggctgtggtacatcagatcttga

SEQ ID NO: 6 - protein, 674 aa, Artificial Sequence
  Amino acid sequence encoding gp140 from HIV Clade A
MRVMEIQRNCQHLLRWGIMILGMIIICSTADNLWVTVYYGVPVWRDAETTLFCASDAKAYSTEKHNVWATHACVPTDPNPQEIPLDNVTEEFNMWKNNMVDQMHEDIISLWDQSLKPCVQLTPLCVTLNCSNARVNATFNSTEDREGMKNCSFNMTTELRDKKQQVYSLFYRLDIEKINSSNNNSEYRLVNCNTSAITQACPKVTFEPIPIHYCAPAGFAILKCNDTEFNGTGPCKNVSTVQCTHGIKPVVSTQLLLNGSLAEREVRIRSENIANNAKNIIVQFASPVKINCIRPNNNTRKSYRIGPGQTFYATDIVGDIRQAHCNVSRTDWNNTLRLVANQLRKYFSNKTIIFTNSSGGDLEITTHSFNCGGEFFYCNTSGLFNSTWTTNNMQESNDTSNGTITLPCRIKQIIRMWQRVGQAMYAPPIEGVIRCESNITGLILTRDGGNNNSANETFRPGGGDIRDNWRSELYKYKVVKIEPLGVAPTRAKRRVVEREKRAVGIGAVFLGFLGAAGSTMGAASITLTVQARQLLSGIVQQQSNLLRAIEAQQQLLKLTVWGIKQLQARVLAVERYLRDQQLLGIWGCSGKLICTTNVPWNSSWSNKSYDDIWQNMTWLQWDKEISNYTDIIYSLIEESQNQQEKNEQDLLALDKWANLWNWFDISKWLWYIRS

SEQ ID NO: 7 - DNA, 1545 bp, Artificial Sequence
  cDNA sequence encoding gp120 from HIV Clade B
atgaaagtgaaggagaccaggaagaattatcagcacttgtggagatggggcaccatgctccttgggatgttgatgatctgtagtgctgcagaacaattgtgggtcacagtctattatggggtacctgtgtggaaagaagcaactaccactctattctgtgcatcagatgctaaagcatatgatacagaggtacataatgtttgggccacacatgcctgtgtacccacagaccccaacccacaagaagtagtattgggaaatgtgacagaatattttaacatgtggaaaaataacatggtagaccagatgcatgaggatataatcagtttatgggatcaaagcttgaagccatgtgtaaaattaaccccactctgtgttactttagattgcgatgatgtgaataccactaatagtactactaccactagtaatggttggacaggagaaataaggaaaggagaaataaaaaactgctcttttaatatcaccacaagcataagagataaggttcaaaaagaatatgcacttttttataaccttgatgtagtaccaatagatgatgataatgctactaccaaaaataaaactactagaaactttaggttgatacattgtaactcctcagtcatgacacaggcctgtccaaaggtatcatttgaaccaattcccatacattattgtgccccggctggttttgcgattctgaagtgtaacaataagacgtttgatggaaaaggactatgtacaaatgtcagcacagtacaatgtacacatggaattaggccagtagtgtcaactcaactgctgttaaatggcagtctagcagaagaagaggtagtaattagatctgacaatttcatggacaatactaaaaccataatagtacagctgaatgaatctgtagcaattaattgtacaagacccaacaacaatacaagaaaaggtatacatataggaccagggagagccttttatgcagcaagaaaaataataggagatataagacaagcacattgtaaccttagtagagcacaatggaataacactttaaaacagatagttataaaattaagagaacactttgggaataaaacaataaaatttaatcaatcctcaggaggggacccagaaattgtaaggcatagttttaattgtggaggggaatttttctactgtgatacaacacaactgtttaatagtacttggaatggtactgaaggaaataacactgaaggaaatagcacaatcacactcccatgtagaataaaacaaattataaacatgtggcaggaagtaggaaaagcaatgtatgcccctcccatcggaggacaaattagatgttcatcaaatattacagggctgctattaacaagagatggtggtaccgaagggaatgggacagagaatgagacagagatcttcagacctggaggaggagatatgagggacaattggagaagtgaattatataaatataaagtagtaaaagttgaaccactaggagtagcacccaccagggcaaagagaagagtggtgcagagataa

SEQ ID NO: 8 - protein, 514 aa, Artificial Sequence
  Amino acid sequence encoding gp120 from HIV Clade B
MKVKETRKNYQHLWRWGTMLLGMLMICSAAEQLWVTVYYGVPVWKEATTTLFCASDAKAYDTEVHNVWATHACVPTDPNPQEVVLGNVTEYFNMWKNNMVDQMHEDIISLWDQSLKPCVKLTPLCVTLDCDDVNTTNSTTTTSNGWTGEIRKGEIKNCSFNITTSIRDKVQKEYALFYNLDVVPIDDDNATTKNKTTRNFRLIHCNSSVMTQACPKVSFEPIPIHYCAPAGFAILKCNNKTFDGKGLCTNVSTVQCTHGIRPVVSTQLLLNGSLAEEEVVIRSDNFMDNTKTIIVQLNESVAINCTRPNNNTRKGIHIGPGRAFYAARKIIGDIRQAHCNLSRAQWNNTLKQIVIKLREHFGNKTIKFNQSSGGDPEIVRHSFNCGGEFFYCDTTQLFNSTWNGTEGNNTEGNSTITLPCRIKQIINMWQEVGKAMYAPPIGGQIRCSSNITGLLLTRDGGTEGNGTENETEIFRPGGGDMRDNWRSELYKYKVVKVEPLGVAPTRAKRRVVQR

SEQ ID NO: 9 - DNA, 2178 bp, Artificial Sequence
  cDNA sequence encoding M72 fusion protein from TB
atgcatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgatgcgggcgggcccgtcgtcaacggcctaggacaggtggtcggtatgaacacggccgcgtcctag

SEQ ID NO: 10 - protein, 709 aa, Artificial Sequence
  Amino acid sequence encoding M72 fusion protein from TB
QGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 11 - DNA, 1149 bp, Artificial Sequence
  cDNA sequence encoding CS protein-derived antigen from P. falciparum
atgatgagaaaacttgccatcctcagcgtcagctctttcctgttcgtggaggccctcttccaggagtatcagtgctacggaagcagcagcaatacaagggtcctgaacgagctcaactatgacaacgctggaacgaacctgtataacgagctggagatgaactactatggcaagcaggagaactggtatagcctgaagaagaacagccggtccctgggcgagaacgacgacggcaacaacaacaacggcgacaacggcagggagggcaaagatgaggacaagagggacgggaacaacgaggataacgagaagctgcggaagcccaagcacaagaaactcaagcagcccgccgacgggaacccggaccccaatgcaaatcccaacgtcgacccaaacgcaaaccctaacgtggaccccaacgccaatcccaacgtcgatcctaatgccaatccaaatgccaaccctaacgcaaatcctaatgcaaaccccaacgccaatcctaacgccaacccaaatgccaacccaaacgctaaccccaacgctaacccaaatgcaaatcccaatgctaacccaaacgtggaccctaacgctaaccccaacgcaaaccctaacgccaatcctaacgcaaaccccaatgcaaacccaaacgcaaatcccaacgctaaccctaacgcaaaccccaacgccaaccctaatgccaaccccaatgctaaccccaacgccaatccaaacgcaaatccaaacgccaacccaaatgcaaaccccaacgctaatcccaacgccaacccaaacgccaatcctaacaagaacaatcagggcaacgggcagggccataacatgccgaacgaccctaatcggaatgtggacgagaacgccaacgccaacagcgccgtgaagaacaacaacaacgaggagccctccgacaagcacatcaaggaatacctgaacaagatccagaacagtctgagcaccgagtggtccccctgctccgtgacctgcggcaacggcatccaggtgaggatcaagcccggctccgccaacaagcccaaggacgagctggactacgccaacgacatcgagaagaagatctgcaagatggagaaatgcagctctgtgttcaacgtcgtgaactccgccatcggcctgtga

SEQ ID NO: 12 - protein, 382 aa, Artificial Sequence
  Amino acid sequence encoding CS protein-derived antigen from P. falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPADGNPDPNANPNVDPNANPNVDPNANPNVDPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSAIGL

SEQ ID NO: 13 - DNA, 1275 bp, Artificial Sequence
  cDNA sequence encoding CS protein-derived fusion protein “RTS” from P. falciparum
atgatggctcccgatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattagggcctgtgacgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaa

SEQ ID NO: 14 - protein, 424 aa, Artificial Sequence
  Amino acid sequence encoding CS protein-derived fusion protein “RTS” from P. falciparum
MMAPDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLGPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 15 - DNA, 3411 bp, Artificial Sequence
  cDNA sequence encoding HIV antigens p24-RT-Nef-p17
atggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgcgatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgaaaaagaaggcccagcaggccgccgccgacaccgggcacagcaaccaggtgtcccaaaactactaa

SEQ ID NO: 16 - protein, 1136 aa, Artificial Sequence
  Amino acid sequence encoding HIV antigens p24-RT-Nef-p17
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSCMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

(19) United States
(12) Patent Application Publication
(10) Pub. No.: US 2011/0236468 A1
(43) Pub. Date: Sep. 29, 2011

(54) VACCINE COMPOSITIONS

(76) Inventors: Clarisse Marie-Madeleine Lorin, Rixensart (BE); Michele Fevrier, Paris (FR); Gerald Hermann Voss, Rixendart (BE); Frederic Tangy, Paris (FR)

(21) Appl. No.: 13/060,823

(22) PCT Filed: Aug. 28, 2009
(86) PCT No.: PCT/EP2009/061105

§ 371 Date: May 26, 2011

(30) Foreign Application Priority Data
Sep. 1, 2008 (GB) 0815872.7

(51) Int. Cl. A61K 9/127; A61K 39/21; A61P 31/18; A61K 39/015; A61P 33/06; A61K 39/04; A61P 31/06

(52) U.S. Cl. 424/450; 424/199.1

(57) ABSTRACT

The present invention relates, inter alia, to a method of raising an immune response against a pathogen which comprises administering (i) one or more first immunogenic polypeptides derived from said pathogen; (ii) one or more viral vectors comprising one or more heterologous polynucleotides encoding one or more second immunogenic polypeptides derived from said pathogen; and (iii) an adjuvant; wherein the one or more first immunogenic polypeptides, the one or more viral vectors and the adjuvant are administered concomitantly. The invention also relates to vaccines, pharmaceutical compositions, kits and uses employing said polypeptides, viral vectors and adjuvants.

### FIELD OF THE INVENTION

This invention relates to novel vaccine compositions and their use in the stimulation of immune responses in mammals, especially humans, and in particular for the prevention and treatment of infection by pathogens. In particular it relates to compositions capable of inducing CD4+ and CD8+ T-cell responses as well as antibody responses in subjects without recourse to complex sequential prime-boost schedules.

### BACKGROUND TO THE INVENTION

Inactivated whole organisms have been used in successful vaccination since the late nineteenth century. In more recent times, vaccines involving the administration of extracts, subunits, toxoids and capsular polysaccharides have been employed. Since genetic engineering techniques have been available, the use of recombinant proteins has been a favoured strategy, obviating many of the risks associated with use of purified proteins from natural sources.

Early vaccine approaches were based on the administration of proteins which stimulated some aspect of the immune response in vivo. Subsequently it was appreciated that immune responses could also be raised by administration of DNA which could be transcribed and translated by the host into an immunogenic protein.

The mammalian immune response has two key components: the humoral response and the cell-mediated response. The humoral response involves the generation of circulating antibodies which will bind to the antigen to which they are specific, thereby neutralising the antigen and favouring its subsequent clearance by a process involving other cells that are either cytotoxic or phagocytic. B-cells are responsible for generating antibodies (plasma B cells), as well as holding immunological humoral memory (memory B-cells), i.e. the ability to recognise an antigen some years after first exposure to it, for example, through vaccination. The cell-mediated response involves the interplay of numerous different types of cells, among which are the T cells. T cells are divided into a number of different subsets, mainly the CD4+ and CD8+ T cells.

Antigen-presenting cells (APC) such as macrophages and dendritic cells act as sentinels of the immune system, screening the body for foreign antigens. When extracellular foreign antigens are detected by APC, these antigens are phagocytosed (engulfed) inside the APC where they will be processed into smaller peptides. These peptides are subsequently presented on major histocompatibility complex class II (MHC II) molecules at the surface of the APC where they can be recognised by antigen-specific T lymphocytes expressing the CD4 surface molecules (CD4+ T cells). When CD4+ T cells recognise the antigen to which they are specific on MHCII molecules in the presence of additional adequate co-stimulatory signals, they become activated and secrete an array of cytokines that subsequently activate the other arms of the immune system. In general, CD4+ T cells are classified into T helper 1 (Th1) or T helper 2 (Th2) subsets depending on the type of response they generate following antigen recognition. Upon recognition of a peptide-MHC II complex, Th1 CD4+ T cells secrete interleukins and cytokines such as interferon gamma thereby activating macrophages to release toxic chemicals such as nitric oxide and reactive oxygen/nitrogen species. IL-2 and TNF-alpha are also commonly categorized as Th1 cytokines. In contrast, Th2 CD4+ T cells generally secrete interleukins such as IL-4, IL-5 or IL-13.

Other functions of the helper CD4+ T cells include providing help to activate B cells to produce and release antibodies. They can also participate to the activation of antigen-specific CD8+ T cells, the other major T cell subset beside CD4+ T cells.

CD8+ T cells recognize the peptide to which they are specific when it is presented on the surface of a host cell by major histocompatibility class I (MHC I) molecules in the presence of appropriate costimulatory signals. In order to be presented on MHC I molecules, a foreign antigen needs to directly access the inside of the cell (the cytosol or nucleus) such as it is the case when a virus or intracellular bacteria directly penetrate a host cell or after DNA vaccination. Inside the cell, the antigen is processed into small peptides that will be loaded onto MHC I molecules that are redirected to the surface of the cell. Upon activation CD8+ T cells secrete an array of cytokines such as interferon gamma that activates macrophages and other cells. In particular, a subset of these CD8+ T cells secretes lytic and cytotoxic molecules (e.g. granzyme, perforin) upon activation. Such CD8+ T cells are referred to as cytotoxic T cells.

More recently, an alternative pathway of antigen presentation involving the loading of extracellular antigens or fragments thereof onto MHCI complexes has been described and called “cross-presentation”.

The nature of the T-cell response is also influenced by the composition of the adjuvant used in a vaccine. For instance, adjuvants containing MPL & QS21 have been shown to activate Th1 CD4+ T cells to secrete IFN-gamma (Stewart et al. Vaccine. 2006, 24 (42-43):6483-92).

Whereas adjuvants are well known to have value in enhancing immune responses to protein antigens, they have not generally been used in conjunction with DNA or DNA-based vector vaccination. There are several hypotheses as to why adjuvants have not been used in conjunction with DNA-vector based vaccines. Indeed, interferences between the adjuvant and the vector may have an impact on their stability. In addition, one might expect that adding an adjuvant to an attenuated vector could increase the reactogenicity induced by such product. Finally, increasing the immunogenicity of a DNA-vector based vaccine may lead to an enhanced neutralizing immune response against the vector itself, thereby precluding any boosting effect of subsequent injections of the same vector-based vaccine. In fact, in a vaccination protocol directed towards protection against P. falciparum infection, Jones et al (2001, J Infect Diseases 183, 303-312) have reported an adverse outcome after combining DNA, recombinant protein and adjuvant as a boosting composition following a prime by DNA. Indeed, the levels of parasitemia were significantly lower in a group in which the boosting composition contained protein and adjuvant only. It was concluded that use of the combination of DNA, recombinant protein and adjuvant in this protocol adversely affected the outcome on parasitemia as well as antibody responses.

On the other hand, there has been a report of enhancement of the efficacy of an adjuvanted DNA-based vector vaccine (Ganne et al. Vaccine (1994) 12(13) 1190-1196). In particular, the enhanced efficacy of a replication-defective adenovirus-vectored vaccine by the addition of oil adjuvants was correlated with higher antibody levels but the impact on CD4+ and CD8+ T cell responses was not reported.

The use of a pathogenic virus as an adjuvant has been disclosed in WO2007/016715. It was not mentioned that said virus could contain any heterologous polynucleotide.

It is generally thought that stimulation of both CD4+ and CD8+ cells are needed for optimal protective immunity, especially in certain diseases such as HIV infection/AIDS. In order to induce an optimal immune response either prophylactically or therapeutically, stimulation of both CD4+ and CD8+ cells is desirable. This is one of the main goals of “prime-boost” vaccination strategies in which the alternate administration of protein-based vaccines (inducing mostly CD4+ T cells) with DNA-vector based vaccines, i.e. naked DNA, viral vectors or intracellular bacterial vectors such as listeria, (inducing mostly CD8+ T cells) or vice versa most likely activates both CD4+ and CD8+ T cell responses.

However, although prime-boost vaccine strategies may generally give rise to a greater or more balanced response, the requirement to vaccinate on more than one occasion and certainly on more than two occasions can be burdensome or even unviable, especially in mass immunization programs for the developing world.

Furthermore, as already mentioned above, it is often not possible to boost the viral vector component because of immunity that may have been raised against the vector itself.

Thus the objects of the invention include one or more of the following: (a) to provide a complete vaccination protocol and a vaccine composition which stimulates the production of CD4+ and/or CD8+ cells and/or antibodies and in particular which obviates or mitigates the need for repeated immunizations; (b) to provide a vaccination protocol and a vaccine composition which is as good as, or better, at stimulating production of CD4+ cells and/or CD8+ cells and/or antibodies relative to vaccine compositions containing an immunogenic polypeptide alone or a polynucleotide alone or relative to a conventional prime-boost protocol involving separate administration of immunogenic polypeptide and polynucleotide; (c) to provide a vaccine composition which stimulates or better stimulates Th 1 cell responses; (d) to provide a vaccine composition and vaccination protocol in which required doses of components, especially viral vectors, are minimised; and (e) more generally to provide a useful vaccine composition and vaccination protocol for treatment or prevention of diseases caused by pathogens. By “better stimulates” is meant that the intensity and/or persistence and/or broadness of the response is enhanced.

### SUMMARY OF THE INVENTION

Thus according to the invention there is provided a method of raising an immune response against a pathogen which comprises administering (i) one or more first immunogenic polypeptides derived from said pathogen; (ii) one or more viral vectors comprising one or more heterologous polynucleotides encoding one or more second immunogenic polypeptides derived from said pathogen; and (iii) an adjuvant; wherein the one or more first immunogenic polypeptides, the one or more viral vectors and the adjuvant are administered concomitantly.

According to a specific aspect of the invention there is provided a vaccine composition comprising (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant.

There is also provided an immunogenic composition comprising (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant.

Said vaccines and immunogenic compositions suitably stimulate production of pathogen-specific CD4+ T-cells and/or CD8+ T-cells and/or antibodies.

By “pathogen-specific CD4+ T-cells and/or CD8+ T-cells and/or antibodies” is meant CD4+ T-cells and/or CD8+ T-cells and/or antibodies which specifically recognise the whole pathogen or a part (e.g., an immunogenic subunit) thereof. By “specifically recognise” is meant that the CD4+ T-cells and/or CD8+ T-cells and/or antibodies recognise in an immunospecific rather than a non-specific manner said pathogen (or part thereof).

There is also provided a method of stimulating an immune response in a mammal which comprises administering to a mammal an immunologically effective amount of such a composition.

There is also provided use of such a composition in the manufacture of a medicament for stimulating an immune response in a mammal.

There is also provided such a composition for use in stimulating an immune response in a mammal.

There is also provided a method of stimulating the production of pathogen-specific CD4+ T-cells and/or CD8+ T-cells and/or antibodies in mammals which comprises administering to said mammal (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant; wherein the one or more first immunogenic polypeptides, the one or more viral vectors and the adjuvant are administered concomitantly, for example by administering an immunologically effective amount of an aforesaid composition.

There is also provided use of aforesaid compositions in the manufacture of a medicament for stimulating the production of pathogen specific CD4+ and/or CD8+ cells and/or antibodies in mammals.

For example, production of CD4+ T-cells or CD8+ T-cells or antibodies is stimulated.

Suitably production of 2 and especially 3 of CD4+ T-cells and/or CD8+ T-cells and/or antibodies is stimulated.

Suitably production of CD8+ T-cells is stimulated. Suitably production of CD4+ and CD8+ T-cells is stimulated. Suitably production of CD4+ and CD8+ T-cells and antibodies is stimulated.

Alternatively suitably production of CD4+ T-cells is stimulated. Suitably production of CD4+ T cells and antibodies is stimulated.

Alternatively suitably production of antibodies is stimulated.

The methods of the invention are suitably intended to provide the steps adequate for a complete method for raising an immune response (although the method may, if desired, be repeated). Therefore suitably the methods do not involve use of a priming dose of any immunogenic polypeptide or polynucleotide (e.g. in the form of a vector such as a viral vector) encoding any immunogenic polypeptide.

For example there is provided a method of raising an immune response against a pathogen which consists of (a) administering (i) one or more first immunogenic polypeptides derived from said pathogen; (ii) one or more viral vectors comprising one or more heterologous polynucleotides encoding one or more second immunogenic polypeptides derived from said pathogen; and (iii) an adjuvant; wherein the one or more immunogenic polypeptide, the one or more viral vector and the adjuvant are administered concomitantly; and (b) optionally repeating the step of (a).

The method may be repeated (e.g. repeated once) if a repeat gives rise to an improved immune response. An adequate response, at least as far as a T-cell response is concerned, may be obtained without any need for repetition.

There is also provided a method of raising an immune response against a pathogen which comprises (a) administering (i) one or more first immunogenic polypeptides derived from said pathogen; (ii) one or more viral vectors comprising one or more heterologous polynucleotides encoding one or more second immunogenic polypeptides derived from said pathogen; and (iii) an adjuvant; wherein the one or more first immunogenic polypeptides, the one or more viral vectors and the adjuvant are administered concomitantly; and wherein the method does not involve administering any priming dose of immunogenic polypeptide or polynucleotide encoding immunogenic polypeptide.

There is also provided a kit comprising (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant; and in particular comprising (i) one or more first immunogenic polypeptides derived from a pathogen and an adjuvant; and (ii) one or more second viral vectors comprising one or more heterologous polynucleotides encoding one or more immunogenic polypeptides derived from said pathogen; for use in a method according to the invention.

Compositions and methods of the invention may be useful for the prevention of infection by pathogens in naïve subjects or for the prevention of infection by pathogens in subjects who have previously been exposed to said pathogen, or prevention of re-infection in subjects who have previously been infected by pathogen or treatment of subjects who have been infected by pathogen.

### BRIEF DESCRIPTION OF THE FIGURES

FIG. 1. F4-specific CD4+ and CD8+ T cell responses 7 days after one co-administration.

A. Mice were immunized once with F4co/AS01B (18 μg) intramuscularly and MV1-F4 (10⁶ TCID₅₀) intraperitonealy. B. 7 days post-immunization, splenocytes were stimulated ex vivo (6 hours before addition of the Brefeldin overnight) with 4 pools of peptides covering the F4 sequence (p24, RT, Nef and p17) and the cytokine production was measured by ICS. HIV-specific response is the addition of p24-, RT-, Nef- and p17-specific responses. The % of HIV-specific CD4+ and CD8+ T cells secreting IFN-γ and/or IL-2 is represented for each mouse.

FIG. 2. F4-specific CD4+ and CD8+ T cell responses 7 days after two co-administrations.

A. Mice were immunized twice with F4co/AS01B (9 μg) and MV1-F4 (10⁵ TCID₅₀) intramuscularly at two different sites at days 0 and 28. B. 7 days post-immunization, splenocytes were stimulated ex vivo (6 hours before addition of the Brefeldin overnight) with 4 pools of peptides covering the F4 sequence (p24, RT, Nef and p17) and the cytokine production was measured by ICS. HIV-specific response is the addition of p24-, RT-, Nef- and p17-specific responses. The % of HIV-specific CD4+ and CD8+ T cells secreting IFN-γ and/or IL-2 is represented for each mouse.

FIG. 3. In vitro infectivity of MV1-F4 when incubated with AS01B adjuvant. MV1-F4 virus was incubated with AS01B adjuvant or medium (OptiMEM) for the indicated time at room temperature. Then the viral titers were assessed on Vero cells by end-point serial dilution assay. The viral titers are expressed in TCID₅₀/ml.

FIG. 4. F4-specific CD4+ T-cell response induced in cynomolgus macaques by F4co/AS01B and MV1-F4 independently or in co-administration.

A. Kinetics and frequencies of F4-specific CD4+ T cells induced in cynomolgus macaques. Monkeys were immunised twice at days 0 and 28 with 10 μg of F4co/AS01B (P), or 4.2 Log CCID₅₀ MV1-F4 (M) or the co-administration of both candidates. Fresh PBMCs were stimulated overnight with a pool of peptides covering the F4 sequence and the cytokine production was measured by intracellular staining (7-color ICS). The median values of 10 monkeys/group were plotted over time. B. Frequencies of F4-specific CD4+ T cells for each individual animal at 14 days post-one injection. The frequency of F4-specific CD4+ T elicited in each animal at 14 days post-one injection is represented for each vaccine regimen (P, M or Co-ad). C. Cytokine co-expression profile of F4-specific CD4+ T cells at 14 days post-first and second immunization. The frequency of F4-specific CD4+ CD40L+ T cells expressing at least one, two or three cytokines (IL2, I IFN-γ and TNF-α) has been assessed by ICS at 14 days post-one and two immunizations. Each pie represents the mean of 10 animals.

FIG. 5. F4-specific CD8+ T-cell response induced in cynomolgus macaques by F4co/AS01B and MV1-F4 independently or in co-administration.

A. Kinetics and frequencies of F4-specific CD8+ T cells induced in cynomolgus macaques. Monkeys were immunised twice at days 0 and 28 with 10 μg of F4co/AS01B (P), or 4.2 Log CCID₅₀ MV1-F4 (M) or the co-administration of both candidates. Fresh PBMCs were stimulated overnight with a pool of peptides covering the F4 sequence and the cytokine production was measured by intracellular staining (7-color ICS). The median values of 10 monkeys/group were plotted over time. B. Frequencies of F4-specific CD8+ T cells for each individual animal at 14 days post-one injection. The frequency of F4-specific CD8+ T elicited in each animal at 14 days post-one injection is represented for each vaccine regimen (P, M or Co-ad). C. Cytokine co-expression profile of F4-specific CD8+ T cells at 14 days post-first and second immunization. The frequency of F4-specific CD8+ T cells expressing at least one, two or three cytokines (IL2, I IFN-γ and TNF-α) has been assessed by ICS at 14 days post-one and two immunizations. Each pie represents the mean of 10 animals.

FIG. 6 Kinetics of the anti-MV and anti-F4co antibody responses in cynomolgus macaques

A. Anti-MV humoral response. Monkeys were immunised twice at days 0 and 28 with 10 μg of F4co/AS01B (P), or 4.2 Log CCID₅₀ MV1-F4 (M) or the co-administration of both candidates. The anti-MV humoral response was measured by an ELISA developed to measure anti-MV antibodies in non-human primate sera. OD values obtained for each animal was plotted over time. B. Anti-F4 humoral response. The mid-point titers of anti-F4co antibodies were determined by ELISA over time. Per time point, geometric means of 10 monkeys/group are represented.

### SUMMARY OF SEQUENCE LISTINGS

| | Sequence Identifier |
|---|---|
| Amino acid or polynucleotide description | (SEQ ID No) |
| HIV Gag-RT-Nef (“GRN”) (Clade B) (cDNA) | 1 |
| HIV Gag-RT-Nef (“GRN”) (Clade B) (amino | 2 |
| acid) | |
| HIV Gag-RT-integrase-Nef (“GRIN”) (Clade A) | 3 |
| (cDNA) | |
| HIV Gag-RT-integrase-Nef (“GRIN”) (Clade A) | 4 |
| (amino acid) | |
| HIV gp140 (Clade A) (cDNA) | 5 |
| HIV gp140 (Clade A) (amino acid) | 6 |
| HIV gp120 (Clade B) (cDNA) | 7 |
| HIV gp120 (Clade B) (amino acid) | 8 |
| TB antigens fusion protein M72 (cDNA) | 9 |
| TB antigens fusion protein M72 (amino acid) | 10 |
| P. falciparum CS protein-derived antigen | 11 |
| (cDNA) | |
| P. falciparum CS protein-derived antigen | 12 |
| (amino acid) | |
| P. falciparum CS protein-derived fusion protein | 13 |
| “RTS” (cDNA) | |
| P. falciparum CS protein-derived fusion protein | 14 |
| “RTS” (amino acid) | |
| HIV p24-RT-Nef-p17 (cDNA) | 15 |
| HIV p24-RT-Nef-p17 (amino acid) | 16 |

The above recited sequences may be employed as polypeptides or polynucletides encoding polypeptides for use in exemplary aspects of the invention. Said polypeptides may consist of or comprise the above mentioned sequences. Initial Met residues are optional. N-terminal His residues (including His residues immediately following an initial Met, as in SEQ ID No 10) are optional or an N-terminal His tag of a different length may be employed (e.g. typically up to 6 His residues may be employed to facilitate isolation of the protein). Analogue proteins which have significant sequence identity e.g. greater than 80%, e.g. greater than 90%, e.g. greater than 95%, e.g. greater than 99% sequence identity over the whole length of the reference sequence may be employed, especially when the analogue protein has a similar function and particularly when the analogue protein is similarly immunogenic. For example up to 20, e.g. up to 10, e.g. 1 to 5 amino acid substitutions (e.g. conservative substitutions) may be tolerated. Nucleic acids which differ from those recited above which encode the same proteins, or the aforementioned analogue proteins, may be employed. Sequence identity may be determined by conventional means e.g. using BLAST. In one specific variant of SEQ ID No 16 that may be mentioned, Cys at position 398 is replaced by Ser.

### DETAILED DESCRIPTION OF THE INVENTION

As used herein the term “concomitantly” means wherein the one or more immunogenic polypeptides, the one or more viral vectors and the adjuvant are administered within a period of no more than 12 hours, e.g. within a period of no more than 1 hour, typically on one occasion. This may be in the course of a single visit to a health professional, for example the one or more immunogenic polypeptides, the one or more viral vectors and the adjuvant are administered sequentially or simultaneously during the same visit.

As used herein, the term “epitope” refers to an immunogenic amino acid sequence. An epitope may refer to a minimum amino acid sequence of typically 6-8 amino acids which minimum sequence is immunogenic when removed from its natural context, for example when transplanted into a heterologous polypeptide. An epitope may also refer to that portion of a protein which is immunogenic, where the polypeptide containing the epitope is referred to as the antigen (or sometimes “polypeptide antigen”). A polypeptide or antigen may contain one or more (eg 2 or 3 or more) distinct epitopes. The term “epitope” embraces B-cell and T-cell epitopes. The term “T-cell epitope” embraces CD4+ T-cell epitopes and CD8+ T-cell epitopes (sometimes also referred to as CTL epitopes).

The term “immunogenic polypeptide” refers to a polypeptide which is immunogenic, that is to say it is capable of eliciting an immune response in an animal, and therefore contains one or more epitopes (eg T-cell and/or B-cell epitopes). Immunogenic polypeptides may contain one or more polypeptide antigens. These may be in a natural or an unnatural arrangement, such as in a fusion protein.

Immunogenic polypeptides will typically be recombinant proteins produced eg by expression in a heterologous host such as a bacterial host, in yeast or in cultured mammalian cells.

The term “polypeptide derived from a pathogen” means a polypeptide which partially or wholly contains sequences (i.e. antigens) which occur naturally in pathogens or bear a high degree of sequence identity thereto (eg more than 95% identity over a stretch of at least 10 eg at least 20 amino acids).

Immunogenic polypeptides may contain one or more (eg 1, 2, 3 or 4) polypeptide antigens.

References herein to polypeptides, antigens, epitopes and polynucleotides include references to fragments or portions thereof.

Unless otherwise specified, an “immune response” may be a cellular and/or a humoral immune response.

In one embodiment of the invention one or more of said one or more first immunogenic polypeptides is substantially the same as one or more of said one or more second immunogenic polypeptides. For example one of the at least one first immunogenic polypeptides and one of the at least one second immunogenic polypeptides may have an overall sequence identity of 90% or more, e.g. 95% or more, e.g. 98% or more, or e.g. 99% or more over the length of one or other immunogenic polypeptides.

In another embodiment of the invention one or more of said one or more first immunogenic polypeptides contains at least one antigen which is substantially the same as an antigen contained in one or more of said one or more second immunogenic polypeptides. For example one of the at least one first immunogenic polypeptides and one of the at least one second immunogenic polypeptides may have an overall sequence identity of 90% or more, e.g. 95% or more, e.g. 98% or more, or e.g. 99% or more over a stretch of 20 amino acids or more, e.g. 40 amino acids or morem e.g. 60 amino acids or more.

Suitably the one or more first immunogenic polypeptides comprise at least one T cell epitope.

Suitably the one or more second immunogenic polypeptides comprise at least one T cell epitope.

Suitably the one or more first immunogenic polypeptides comprise at least one B cell epitope.

Suitably the one or more second immunogenic polypeptides comprise at least one B cell epitope

In another embodiment of the invention one or more of said one or more first immunogenic polypeptides and one or more of said one or more second immunogenic polypeptides share one or more identical B-cell and/or T-cell epitopes. Suitably they share one or more identical amino acid sequences of length 10 amino acids or more, e.g. 15 amino acids or more, e.g. 25 amino acids or more.

In another embodiment of the invention, none of the one or more of said one or more first immunogenic polypeptides is substantially the same as or contains any antigen in common with one or more of said one or more second immunogenic polypeptides, for example they may have an overall sequence identity of less than 90% over a stretch of 20 amino acids or more, e.g. 40 amino acids or more, e.g. 60 amino acids or more.

Thus, they may not share any B-cell or T-cell epitopes. For example, they may not share any identical amino acid sequences of length 10 amino acids or more, e.g. at 15 amino acids or more, e.g. 25 amino acids or more.

In one specific embodiment of the invention a first immunogenic polypeptide and a second immunogenic polypeptide contain the same antigens in the same arrangement or in a different arrangement. By “different arrangement” is meant that they may be arranged in a different order and/or they may be divided, for example an antigen may be split and arranged either side of another antigen or antigens. In such example, an antigen may be split at any point along its length. In another specific embodiment of the invention a first immunogenic polypeptide and a second immunogenic polypeptide are the same.

The composition according to the invention may contain one first immunogenic polypeptide as the only immunogenic polypeptide in the composition. Alternatively the composition according to the invention may contain more than one first immunogenic polypeptides, e.g. 2 or 3 or 4 or more immunogenic polypeptides.

The composition according to the invention may comprise one viral vector. Alternatively it may comprise more than one viral vector, e.g. 2 or more viral vectors.

In compositions according to the invention a viral vector may comprise a heterologous polynucleotide which encodes for one second immunogenic polypeptide or it may comprise more than one heterologous polynucleotide which together encode for more than one second immunogenic polypeptide, which may be under the control of the same promoter or more than one promoter.

As well as for prophylactic vaccination, the compositions of the invention may also be used in individuals that are already infected with a pathogen, and result in improved immunological control or clearance of the established infection. This is of particular interest when the pathogen is HIV. In the case of HIV, this control is believed to be achieved by CD8-positive T cells that specifically recognize HIV-infected cells. Such CD8-positive T cell response is maintained by the presence of HIV-specific CD4-positive helper T cells. Therefore, the induction of both types of immune response is particularly useful, and can be achieved by combining different vaccine compositions. A combination of an adjuvanted protein and a recombinant virus is of particular interest. The HIV-infected patients that will benefit from the above-described vaccination are either in the primary infection, latency or terminal phase of HIV infection at the time of vaccination. The patients may or may not undergo other therapeutic treatment interventions against pathogen (in the case of HIV—for example highly active antiretroviral therapy) at the time of vaccination, or at a time close to vaccination.

### Antigens

Antigens of use according to the invention are derived from pathogens. Pathogens include viruses, bacteria, protozoa and other parasitic organisms harmful to animals including man.

Suitable polypeptide antigens to be administered as polypeptide or polynucleotide encoding polypeptide according to the invention include antigens derived from HIV (eg HIV-1), human herpes viruses (such as gH, gL gM gB gC gK gE or gD or derivatives thereof or Immediate Early protein such as ICP27, ICP 47, ICP4, ICP36 from HSV1 or HSV2), cytomegalovirus, especially Human, (such as gB or derivatives thereof), Epstein Barr virus (such as gp350 or derivatives thereof), Varicella Zoster Virus (such as gpl, II, III and IE63), or from a hepatitis virus such as hepatitis B virus (for example Hepatitis B Surface antigen, PreS1, PreS2 and Surface env proteins, Hepatitis B core antigen or pol), hepatitis C virus (eg Core, E1, E2, P7, NS2, NS3, NS4A, NS4B, NS5A and B) and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus (such as haemaggluttin, nucleoprotein, NA, or M proteins, or combinations thereof), or antigens derived from bacterial pathogens such as Neisseria spp, including N. gonorrhea and N. meningitidis, eg, transferrin-binding proteins, lactoferrin binding proteins, PiIC, adhesins); S. pyogenes (for example M proteins or fragments thereof, C5A protease), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example anthrax toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis, C. pneumoniae, C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum and P. vivax; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; leishmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Further bacterial antigens include antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial antigens include antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

In particular, the methods or compositions of the present invention may be used to protect against or treat viral disorders such as those caused by Hepatitis B virus, Hepatitis C virus, Human papilloma virus, Human immunodeficiency virus (HIV), or Herpes simplex virus; bacterial diseases such as those caused by Mycobacterium tuberculosis (TB) or Chlamydia sp; and protozoal infections such as malaria.

It is to be recognised that these specific disease states, pathogens and antigens have been referred to by way of example only, and are not intended to be limiting upon the scope of the present invention.

### TB Antigens

The pathogen may, for example, be Mycobacterium tuberculosis.

Exemplary antigens derived from M. tuberculosis are for example alpha-crystallin (HspX), HBHA, Ry1753, Rv2386, Rv2707, Rv2557, Rv2558, RPFs: Rv0837c, Rv1884c, Rv2389c, Rv2450, Ry1009, aceA (Rv0467), ESAT6, Tb38-1, Ag85A, -B or -C, MPT 44, MPT59, MPT45, HSP10, HSP65, HSP70, HSP 75, HSP90, PPD 19 kDa [Rv3763], PPD, 38 kDa [Rv0934]), PstS1, (Rv0932), SodA (Rv3846), Rv2031c, 16 kDa, Ra12, TbH9, Ra35, Tb38-1, Erd 14, DPV, MTI, MSL, DPPD, mTCC1, mTCC2, hTCC1 (WO 99/51748) and hTCC2, and especially Mtb32a, Ra35, Ra12, DPV, MSL, MTI, Tb38-1, mTCC1, TbH9 (Mtb39a), hTCC1, mTCC2 and DPPD. Antigens derived from M. tuberculosis also include fusion proteins and variants thereof where at least two, or for example, three polypeptides of M. tuberculosis are fused into a larger protein. Such fusions may comprise or consist of Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748), Ra12-Tbh9-Ra35-Ag85B and Ra12-Tbh9-Ra35-mTCC2. A particular Ra12-Tbh9-Ra35 sequence that may be mentioned is defined by SEQ ID No 6 of WO2006/117240 together with variants in which Ser 704 of that sequence is mutated to other than serine, eg to Ala, and derivatives thereof incorporating an N-terminal His tag of an appropriate length (eg SEQ ID No 2 or 4 of WO2006/117240). See also SEQ ID No 10 which is a sequence containing an optional starting M and an optional N-terminal His-His tag (positions 2 and 3) and in which the Ala mutated relative to the wild-type Ser is at position 706.

### Chlamydia Antigens

The pathogen may, for example, be a Chlamydia sp. eg C. trachomatis. Exemplary antigens derived from Chlamydia sp eg C. trachomatis are selected from CT858, CT089, CT875, MOMP, CT622, PmpD, PmpG and fragments thereof, SWIB and immunogenic fragments of any one thereof (such as PmpDpd and PmpGpd) and combinations thereof. Preferred combinations of antigens include CT858, CT089 and CT875. Specific sequences and combinations that may be employed are described in WO2006/104890.

### Plasmodium Antigens

The pathogen may, for example be a parasite that causes malaria such as a Plasmodium sp. eg P. falciparum or P. vivax.

For example, antigens derived from P. falciparum include circumsporozoite protein (CS protein), PfEMP-1, Pfs 16 antigen, MSP-1, MSP-3, LSA-1, LSA-3, AMA-1 and TRAP. A particular hybrid antigen that may be mentioned is RTS. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. When expressed in yeast RTS is produced as a lipoprotein particle, and when it is co-expressed with the S antigen from HBV it produces a mixed particle known as RTS,S The structure or RTS and RTS,S is disclosed in WO 93/10152. TRAP antigens are described in WO 90/01496. Other Plasmodium antigens include P. falciparum EBA, GLURP, RAP1, RAP2, Sequestrin, Pf332, STARP, SALSA, PfEXP1, Pfs25, Pfs28, PFS27/25, Pfs48/45, Pfs230 and their analogues in other Plasmodium spp. One embodiment of the present invention is a composition comprising RTS,S or CS protein or a fragment thereof such as the CS portion of RTS,S in combination with one or more further malarial antigens which may be selected for example from the group consisting of MSP-1, MSP-3, AMA-1, Pfs 16, LSA-1 or LSA-3. Possible antigens from P. vivax include circumsporozoite protein (CS protein) and Duffy antigen binding protein and immunogenic fragments thereof, such as PvRII (see eg WO02/12292).

Thus in one suitable embodiment of the invention, the first and second immunogenic polypeptides are selected from antigens derived from Plasmodium falciparum and/or Plasmodium vivax.

For example, the first and/or second immunogenic polypeptides are selected from antigens derived from Plasmodium falciparum and/or Plasmodium vivax and are selected from RTS (eg as RTS,S), circumsporozoite (CS) protein, MSP-1, MSP-3, AMA-1, LSA-1, LSA-3 and immunogenic derivatives thereof or immunogenic fragments thereof.

One specific derivative that may be mentioned is the hybrid protein known as RTS, especially when presented in the form of a mixed particle known as RTS,S.

An exemplary RTS sequence is shown in SEQ ID No 14.

An exemplary P. falciparum CS protein-derived antigen is shown in SEQ ID No 12. This particular sequence corresponds to the CSP sequence of P. falciparum (3D7 strain), which also contains a 19 aa insertion coming from 7G8 strain (81-100).

In one specific embodiment of the invention, a first immunogenic polypeptide is RTS,S and a second immunogenic polypeptide is the CS protein from Plasmodium falciparum or an immunogenic fragment thereof.

### HPV Antigens

The pathogen may, for example, be a Human Papilloma Virus.

Thus antigens of use in the present invention may, for example, be derived from the Human Papilloma Virus (HPV) considered to be responsible for genital warts (HPV 6 or HPV 11 and others), and/or the HPV viruses responsible for cervical cancer (HPV16, HPV18, HPV33, HPV51, HPV56, HPV31, HPV45, HPV58, HPV52 and others). In one embodiment the forms of genital wart prophylactic, or therapeutic, compositions comprise L1 particles or capsomers, and fusion proteins comprising one or more antigens selected from the HPV proteins E1, E2, E5 E6, E7, L1, and L2. In one embodiment the forms of fusion protein are: L2E7 as disclosed in WO96/26277, and proteinD (1/3)-E7 disclosed in PCT/EP98/05285.

A preferred HPV cervical infection or cancer, prophylaxis or therapeutic composition may comprise HPV 16 or 18 antigens. For example, L1 or L2 antigen monomers, or L1 or L2 antigens presented together as a virus like particle (VLP) or the L1 protein presented alone in a VLP or capsomer structure. Such antigens, virus like particles and capsomer are per se known. See for example WO94/00152, WO94/20137, WO94/05792, and WO93/02184. Additional early proteins may be included alone or as fusion proteins such as E7, E2 or preferably E5 for example; particularly preferred embodiments of this invention include a VLP comprising L1E7 fusion proteins (WO 96/11272). In one embodiment the HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277). Alternatively the HPV 16 or 18 early proteins E6 and E7, may be presented in a single molecule, preferably a Protein D-E6/E7 fusion. Such a composition may optionally provide either or both E6 and E7 proteins from HPV 18, preferably in the form of a Protein D-E6 or Protein D-E7 fusion protein or Protein D E6/E7 fusion protein. Additionally antigens from other HPV strains, preferably from strains HPV 31 or 33 may be employed.

### HIV Antigens

The pathogen may, for example, be HIV, e.g. HIV-1.

Thus, antigens may be selected from HIV derived antigens, particularly HIV-1 derived antigens.

HIV Tat and Nef proteins are early proteins, that is, they are expressed early in infection and in the absence of structural proteins.

The Nef gene encodes an early accessory HIV protein which has been shown to possess several activities. For example, the Nef protein is known to cause the removal of CD4, the HIV receptor, from the cell surface, although the biological importance of this function is debated. Additionally Nef interacts with the signal pathway of T cells and induces an active state, which in turn may promote more efficient gene expression. Some HIV isolates have mutations or deletions in this region, which cause them not to encode functional protein and are severely compromised in their replication and pathogenesis in vivo.

The Gag gene is translated from the full-length RNA to yield a precursor polyprotein which is subsequently cleaved into 3-5 capsid proteins; the matrix protein p17, capsid protein p24 and nucleic acid binding protein (Fundamental Virology, Fields B N, Knipe D M and Howley M 1996 2. Fields Virology vol 2 1996).

The Gag gene gives rise to the 55-kilodalton (Kd) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the Pol gene) during the process of viral maturation into four smaller proteins designated MA (matrix [p17]), CA (capsid [p24]), NC (nucleocapsid [p9]), and p6.

In addition to the 3 major Gag proteins (p17, p24 and p9), all Gag precursors contain several other regions, which are cleaved out and remain in the virion as peptides of various sizes. These proteins have different roles e.g. the p2 protein has a proposed role in regulating activity of the protease and contributes to the correct timing of proteolytic processing.

The MA polypeptide is derived from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV to infect non-dividing cells, an unusual property for a retrovirus.

The p24 (CA) protein forms the conical core of viral particles. Cyclophilin A has been demonstrated to interact with the p24 region of p55 leading to its incorporation into HIV particles. The interaction between Gag and cyclophilin A is essential because the disruption of this interaction by cyclosporin inhibits viral replication.

The NC region of Gag is responsible for specifically recognizing the so-called packaging signal of HIV. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

The Pol gene encodes three proteins having the activities needed by the virus in early infection reverse transcriptase RT, protease, and the integrase protein needed for integration of viral DNA into cellular DNA. The primary product of Pol is cleaved by the virion protease to yield the amino terminal RT peptide which contains activities necessary for DNA synthesis (RNA and DNA directed DNA polymerase, ribonuclease H) and carboxy terminal integrase protein. HIV RT is a heterodimer of full-length RT (p66) and a cleavage product (p51) lacking the carboxy terminal RNase H domain.

RT is one of the most highly conserved proteins encoded by the retroviral genome. Two major activities of RT are the DNA Pol and ribonuclease H activity. The DNA Pol activity of RT uses RNA and DNA as templates interchangeably and, like all DNA polymerases known, is unable to initiate DNA synthesis de novo, but requires a pre-existing molecule to serve as a primer (RNA).

The RNase H activity inherent in all RT proteins plays the essential role early in replication of removing the RNA genome as DNA synthesis proceeds. It selectively degrades the RNA from all RNA-DNA hybrid molecules. Structurally the polymerase and ribo H occupy separate, non-overlapping domains within the Pol covering the amino two thirds of the Pol.

The p66 catalytic subunit is folded into 5 distinct subdomains. The amino terminal 23 of these have the portion with RT activity. Carboxy terminal to these is the RNase H domain.

After infection of the host cell, the retroviral RNA genome is copied into linear double stranded DNA by the reverse transcriptase that is present in the infecting particle. The integrase (reviewed in Skalka A M '99 Adv in Virus Res 52 271-273) recognises the ends of the viral DNA, trims them and accompanies the viral DNA to a host chromosomal site to catalyse integration. Many sites in the host DNA can be targets for integration. Although the integrase is sufficient to catalyse integration in vitro, it is not the only protein associated with the viral DNA in vivo—the large protein-viral DNA complex isolated from the infected cells has been denoted the pre integration complex. This facilitates the acquisition of the host cell genes by progeny viral genomes.

The integrase is made up of 3 distinct domains, the N terminal domain, the catalytic core and the C terminal domain. The catalytic core domain contains all of the requirements for the chemistry of polynucleotidyl transfer.

HIV-1 derived antigens for use in the invention may thus for example be selected from Gag (for example full length Gag), p17 (a portion of Gag), p24 (another portion of Gag), p41, p40, Pol (for example full length Pol), RT (a portion of Pol), p51 (a portion of RT), integrase (a portion of Pol), protease (a portion of Pol), Env, gp120, gp140 or gp160, gp41, Nef, Vif, Vpr, Vpu, Rev, Tat and immunogenic derivatives thereof and immunogenic fragments thereof, particularly Env, Gag, Nef and Pol and immunogenic derivatives thereof and immunogenic fragments thereof including p17, p24, RT and integrase. HIV vaccines may comprise polypeptides and/or polynucleotides encoding polypeptides corresponding to multiple different HIV antigens for example 2 or 3 or 4 or more HIV antigens which may be selected from the above list. Several different antigens may, for example, be comprised in a single fusion protein. More than one first immunogenic polypeptide and/or more than one second immunogenic polypeptide each of which is an HIV antigen or a fusion of more than one antigen may be employed.

For example an antigen may comprise Gag or an immunogenic derivative or immunogenic fragment thereof, fused to RT or an immunogenic derivative or immunogenic fragment thereof, fused to Nef or an immunogenic derivative or immunogenic fragment thereof wherein the Gag portion of the fusion protein is present at the 5′ terminus end of the polypeptide.

A Gag sequence of use according to the invention may exclude the Gag p6 polypeptide encoding sequence. A particular example of a Gag sequence for use in the invention comprises p17 and/or p24 encoding sequences.

A RT sequence may contain a mutation to substantially inactivate any reverse transcriptase activity (see WO03/025003).

The RT gene is a component of the bigger pol gene in the HIV genome. It will be understood that the RT sequence employed according to the invention may be present in the context of Pol, or a fragment of Pol corresponding at least to RT. Such fragments of Pol retain major CTL epitopes of Pol. In one specific example, RT is included as just the p51 or just the p66 fragment of RT.

The RT component of the fusion protein or composition according to the invention optionally comprises a mutation to remove a site which serves as an internal initiation site in prokaryotic expression systems.

Optionally the Nef sequence for use in the invention is truncated to remove the sequence encoding the N terminal region i.e. removal of from 30 to 85 amino acids, for example from 60 to 85 amino acids, particularly the N terminal 65 amino acids (the latter truncation is referred to herein as trNef). Alternatively or additionally the Nef may be modified to remove the myristylation site. For example the Gly 2 myristylation site may be removed by deletion or substitution. Alternatively or additionally the Nef may be modified to alter the dileucine motif of Leu 174 and Leu 175 by deletion or substitution of one or both leucines. The importance of the dileucine motif in CD4 downregulation is described e.g. in Bresnahan P. A. et al (1998) Current Biology, 8(22): 1235-8.

The Env antigen may be present in its full length as gp160 or truncated as gp140 or shorter (optionally with a suitable mutation to destroy the cleavage site motif between gp120 and gp41). The Env antigen may also be present in its naturally occurring processed form as gp120 and gp41. These two derivatives of gp160 may be used individually or together as a combination. The aforementioned Env antigens may further exhibit deletions (in particular of variable loops) and truncations. Fragments of Env may be used as well.

An exemplary gp120 sequence is shown in SEQ ID No 8. An exemplary gp140 sequence is shown in SEQ ID No 6.

Immunogenic polypeptides according to the invention may comprise Gag, Pol, Env and Nef wherein at least 75%, or at least 90% or at least 95%, for example, 96% of the CTL epitopes of these native antigens are present.

In immunogenic polypeptides according to the invention which comprise p17/p24 Gag, p66 RT, and truncated Nef as defined above, 96% of the CTL epitopes of the native Gag, Pol and Nef antigens are suitably present.

One embodiment of the invention provides an immunogenic polypeptide containing p17, p24 Gag, p66 RT, truncated Nef (devoid of nucleotides encoding terminal amino-acids 1-85 —“trNef”) in the order Gag, RT, Nef. In polynucleotides encoding immunogenic polypeptides of the invention, suitably the P24 Gag and P66 RT are codon optimized.

Specific polynucleotide constructs and corresponding polypeptide antigens according to the invention include:

1. p17, p24 (codon optimised) Gag-p66 RT (codon optimised)-truncated Nef;
2. truncated Nef-p66 RT (codon optimised)-p17, p24 (codon optimised) Gag;
3. truncated Nef-p17, p24 (codon optimised) Gag-p66 RT (codon optimised);
4. p66 RT (codon optimised)-p17, p24 (codon optimised) Gag-truncated Nef;
5. p66 RT (codon optimised)-truncated Nef-p17, p24 (codon optimised) Gag;
6. p17, p24 (codon optimised) Gag-truncated Nef-p66 RT (codon optimised).

An exemplary fusion is a fusion of Gag, RT and Nef particularly in the order Gag-RT-Nef (see eg SEQ ID No 2). Another exemplary fusion is a fusion of p17, p24, RT and Nef particularly in the order p24-RT-Nef-p17 (see eg SEQ ID No 16, referred to elsewhere herein as “F4”).

In another embodiment an immunogenic polypeptide contains Gag, RT, integrase and Nef, especially in the order Gag-RT-integrase-Nef (see eg SEQ ID No 4).

In other embodiments the HIV antigen may be a fusion polypeptide which comprises Nef or an immunogenic derivative thereof or an immunogenic fragment thereof, and p17 Gag and/or p24 Gag or immunogenic derivatives thereof or immunogenic fragments thereof, wherein when both p17 and p24 Gag are present there is at least one HIV antigen or immunogenic fragment between them.

For example, Nef is suitably full length Nef.

For example p17 Gag and p24 Gag are suitably full length p17 and p24 respectively.

In one embodiment an immunogenic polypeptide comprises both p17 and p24 Gag or immunogenic fragments thereof. In such a construct the p24 Gag component and p17 Gag component are separated by at least one further HIV antigen or immunogenic fragment, such as Nef and/or RT or immunogenic derivatives thereof or immunogenic fragments thereof. See WO2006/013106 for further details.

In fusion proteins which comprise p24 and RT, it may be preferable that the p24 precedes the RT in the construct because when the antigens are expressed alone in E. coli better expression of p24 than of RT is observed.

Some constructs according to the invention include the following:

1. p24-RT-Nef-p17
2. p24-RT*-Nef-p17
3. p24-p51 RT-Nef-p17
4. p24-p51RT*-Nef-p17
5. p17-p51RT-Nef
6. p17-p51RT*-Nef

### 7. Nef-p17

8. Nef-p17 with linker
9. p17-Nef
10. p17-Nef with linker
* represents RT methionine₅₉₂ mutation to lysine

In another aspect the present invention provides a fusion protein of HIV antigens comprising at least four HIV antigens or immunogenic fragments, wherein the four antigens or fragments are or are derived from Nef, Pol and Gag. Preferably Gag is present as two separate components which are separated by at least one other antigen in the fusion. Preferably the Nef is full length Nef. Preferably the Pol is p66 or p51RT. Preferably the Gag is p17 and p24 Gag. Other preferred features and properties of the antigen components of the fusion in this aspect of the invention are as described herein.

Preferred embodiments of this aspect of the invention are the four component fusions as already listed above:

1. p24-RT-Nef-p17
2. p24-RT*-Nef-p17
3. p24-p51 RT-Nef-p17
4. p24-p51RT*-Nef-p17

The immunogenic polypeptides of the present invention may have linker sequences present in between the sequences corresponding to particular antigens such as Gag, RT and Nef. Such linker sequences may be, for example, up to 20 amino acids in length. In a particular example they may be from 1 to 10 amino acids, or from 1 to 6 amino acids, for example 4 to 6 amino acids.

Further description of such suitable HIV antigens can be found in WO03/025003.

HIV antigens of the present invention may be derived from any HIV clade, for example clade A, clade B or clade C. For example the HIV antigens may be derived from clade A or B, especially B.

In one specific embodiment of the invention, a first immunogenic polypeptide is a polypeptide comprising Gag and/or Pol and/or Nef or a fragment or derivative of any of them (eg p24-RT-Nef-p17). In one specific embodiment of the invention a second immunogenic polypeptide is a polypeptide comprising Gag and/or Pol and/or Nef or a fragment or derivative of any of them (eg Gag-RT-Nef or Gag-RT-integrase-Nef).

Thus in one specific embodiment, a polypeptide comprising Gag and/or Pol and/or Nef or a fragment or derivative of any of them (eg p24-RT-Nef-p17) is a first immunogenic polypeptide and a polypeptide comprising Gag and/or Pol and/or Nef or a fragment or derivative of any of them (eg Gag-RT-Nef or Gag-RT-integrase-Nef) is a second immunogenic polypeptide.

In another specific embodiment of the invention, a first immunogenic polypeptide is Env or a fragment or derivative thereof, e.g. gp120, gp140 or gp160 (especially gp120). In one specific embodiment of the invention a second immunogenic polypeptide is a polypeptide comprising Gag and/or Pol and/or Nef or a fragment or derivative of any of them (eg p24-RT-Nef-p17).

Thus in one specific embodiment, Env or a fragment or derivative thereof, e.g. gp120, gp140 or gp160 (especially gp120) is a first immunogenic polypeptide and a polypeptide comprising Gag and/or Pol and/or Nef or a fragment or derivative of any of them (eg p24-RT-Nef-p17) is a second immunogenic polypeptide.

In another specific embodiment of the invention, a first immunogenic polypeptide is a polypeptide comprising Gag and/or Pol and/or Nef or a fragment or derivative of any of them (eg p24-RT-Nef-p17). In one specific embodiment of the invention a second immunogenic polypeptide is Env or a fragment or derivative thereof, e.g. gp120, gp140 or gp160 (especially gp120).

Thus in one specific embodiment, a polypeptide comprising Gag and/or Pol and/or Nef or a fragment or derivative of any of them (eg p24-RT-Nef-p17) is a first immunogenic polypeptide and Env or a fragment or derivative thereof, e.g. gp120, gp140 or gp160 (especially gp120) is a second immunogenic polypeptide.

### Immunogenic Derivatives and Immunogenic Fragments of Antigens

The aforementioned antigens may be employed in the form of immunogenic derivatives or immunogenic fragments thereof rather than the whole antigen.

As used herein the term “immunogenic derivative” in relation to an antigen of native origin refers to an antigen that may have been modified in a limited way relative to its native counterparts. For example it may include a point mutation which may change the properties of the protein, e.g. by improving expression in prokaryotic systems or by removing undesirable activity, e.g. enzymatic activity. Immunogenic derivatives will however be sufficiently similar to the native antigens such that they retain their antigenic properties and remain capable of raising an immune response against the native antigen. Whether or not a given derivative raises such an immune response may be measured by a suitably immunological assay such as an ELISA (for antibody responses) or flow cytometry using suitable staining for cellular markers (for cellular responses).

Immunogenic fragments are fragments which encode at least one epitope, for example a CTL epitope, typically a peptide of at least 8 amino acids. Fragments of at least 8, for example 8 to 10 amino acids or up to 20, 50, 60, 70, 100, 150 or 200 amino acids in length are considered to fall within the scope of the invention as long as the polypeptide demonstrates antigenicity, that is to say that the major epitopes (eg CTL epitopes) are retained by the polypeptide.

### Viral Vectors

Viral vectors of the present invention comprise one or more heterologous polynucleotides which encode one or more immunogenic polypeptides.

The viral vector may be any viral vector, although in one aspect adenoviral vectors are excluded from the scope of the invention.

Viral vectors may be derived from any suitable viral type. Virus types include:
dsDNA viruses (e.g. Adenoviruses, Herpesviruses, Poxviruses)
ssDNA viruses (+) sense DNA (e.g. Parvoviruses)
dsRNA viruses (e.g. Reoviruses)
(+)ssRNA viruses (+) sense RNA (e.g. Picornaviruses, Togaviruses)
(−)ssRNA viruses (−) sense RNA (e.g. Orthomyxoviruses, Rhabdoviruses)
ssRNA-RT viruses (+) sense RNA with DNA intermediate in life-cycle (e.g. Retroviruses)
dsDNA-RT viruses (e.g. Hepadnaviruses)
   DNA virus types include: Adenoviridae; Papillomaviridae; Parvoviridae; Herpesviridae eg Herpes simplex virus, varicella-zoster virus, cytomegalovirus, Epstein-Barr virus; Poxyiridae eg Smallpox virus, vaccinia virus; Hepadnaviridae eg Hepatitis B virus; Polyomaviridae eg Polyoma virus, JC virus (progressive multifocal leucoencephalopathy); Circoviridae eg Transfusion Transmitted Virus.
   RNA virus types include Reoviridae eg Reovirus, Rotavirus; Picornaviridae eg Enterovirus, Rhinovirus, Hepatovirus, Cardiovirus, Aphthovirus, Poliovirus, Parechovirus, Erbovirus, Kobuvirus, Teschovirus, Coxsackie; Caliciviridae eg Norwalk virus, Hepatitis E; Togaviridae eg Rubella virus; Arenaviridae eg Lymphocytic choriomeningitis virus; Flaviviridae eg Dengue virus, Hepatitis C virus, Yellow fever virus; Orthomyxoviridae eg Influenzavirus A, Influenzavirus B, Influenzavirus C, Isavirus, Thogotovirus; Paramyxoviridae eg Measles virus, Mumps virus, Respiratory syncytial virus; Bunyaviridae eg California encephalitis virus, Hantavirus; Rhabdoviridae eg Rabies virus; Filoviridae eg Ebola virus, Marburg virus; Coronaviridae eg Corona virus; Astroviridae eg Astrovirus; Bornaviridae eg Borna disease virus.
   RT virus types include Metaviridae; Pseudoviridae; Retroviridae—eg HIV; Hepadnaviridae—e.g. Hepatitis B virus; Caulimoviridae—e.g. Cauliflower mosaic virus.

The viral vector may be, by way of example, a positive strand RNA virus, for example Retroviridae such as mouse leukemia virus, feline leukemia virus, adult T cell leukemia virus, human immunodeficiency virus, feline immunodeficiency virus and simian immunodeficiency virus; Togaviridae such as alphaviruses including semliki forest virus (SFV), sindbis virus and venezuelan equine encephalitis; flaviviruses including yellow fever virus and rubella virus; and Picornaviridae such as picornavirus.

The viral vector may be, by way of example, a negative strand RNA virus, for example Paramyoxoviridae such as sendai virus, Newcastle disease virus, mumps virus, respiratory syncytial virus, and, in particular, measles virus; Orthomyxoviridae such as influenza virus; or Rhabdoviridae such as vesicular stomatitis virus and rabies virus.

The viral vector may be, by way of example, a single stranded DNA virus belonging to Parvoviridae such as adeno-associated virus.

The viral vector may be, by way of example, a double stranded DNA virus belonging to Herpesviridae such as Epstein-Barr virus, herpes simplex virus (HSV); Poxyiridae such as vaccinia virus and derivatives such as modified vaccinia Ankara (MVA), canarypox and fowlpox.

In one aspect of the invention the vector is the measles virus. Measles virus (MV) belongs to the genus Morbillivirus in the family Paramyxoviridae. The Edmonston strain of MV was isolated in 1954, serially passaged on primary human kidney and amnion cells, and then adapted to chicken embryo fibroblasts (CEF) to produce Edmonston A and B seeds. Edmonston B was licensed in 1963 as the first MV vaccine. Further passages of Edmonston A and B on CEF produced the more attenuated Schwarz and Moraten viruses, whose sequences have recently been shown to be identical. Being reactogenic, Edmonston B vaccine was abandoned in 1975 and was replaced by the Schwarz/Moraten vaccine. This is now the most commonly used measles vaccine. By now, MV vaccine has been given to billions of people and is safe and efficacious. It induces a very efficient, life-long CD4, CD8, and humoral immunity after a single injection of 104 50% tissue culture infective doses (TCI D50). Its safety is due to the fact that the genome is very stable, which explains that reversion to pathogenicity has never been observed, and that it cannot be integrated in host chromosomes, since viral replication is exclusively cytoplasmic.

Measles viral vectors are disclosed in, by way of example, WO2008/078198, WO 2006/136697, WO2004/001051 and WO2004/000876, the Journal of Virology, November 2003, p. 11546-11554, Vol. 77, No. 21, publication entitled “A Molecularly Cloned Schwarz Strain of Measles Virus Vaccine Induces Strong Immune Responses in Macaques and Transgenic Mice”, Chantal Combredet, et al., all herein fully incorporated by reference.

In one aspect the viral vector is an attenuated Schwartz measles strain, for example as disclosed in the above publications.

In one aspect the disclosure relates to the use of a measles vector in combination with HIV antigens, and in particular a measles vector comprising a polynucleotide encoding an HIV polypeptide comprising one or more of Nef, Env, Gag, or RT, either full length or an immunogenic fragment or derivatives thereof.

The viral vector of the invention may be replication defective. This means that it has a reduced ability to replicate in non-complementing cells, compared to the wild type virus. This may be brought about by mutating the virus e.g. by deleting a gene involved in replication.

The viral vectors can be produced on any suitable cell line in which the virus is capable of replication. Where the virus has impaired replication due to missing factors, then complementing cell lines which provide the factors missing from the viral vector that result in its impaired replication characteristics can be used.

The polynucleotide sequences which encode immunogenic polypeptides may be codon optimised for mammalian cells. The principle of such codon-optimisation is described in detail in WO05/025614. Codon optimization for certain HIV sequences is further described in WO 03/025003

In one embodiment of the present invention the polynucleotide constructs comprise an N-terminal leader sequence. The signal sequence, transmembrane domain and cytoplasmic domain are individually all optionally present or deleted. In one embodiment of the present invention all these regions are present but modified.

A promoter for use in the viral vector according to the invention may be the promoter from HCMV IE gene, for example wherein the 5′ untranslated region of the HCMV IE gene comprising exon 1 is included and intron A is completely or partially excluded as described in WO 02/36792.

When several antigens are fused into a fusion protein, such protein would be encoded by a polynucleotide under the control of a single promoter.

In an alternative embodiment of the invention, several antigens may be expressed separately through individual promoters, each of said promoters may be the same or different. In yet another embodiment of the invention some of the antigens may form a fusion, linked to a first promoter and other antigen(s) may be linked to a second promoter, which may be the same or different from the first promoter.

Thus, the viral vector may comprise one or more expression cassettes each of which encode one antigen under the control of one promoter. Alternatively or additionally it may comprise one or more expression cassettes each of which encode more than one antigen under the control of one promoter, which antigens are thereby expressed as a fusion. Each expression cassette may be present in more than one locus in the viral vector.

The polynucleotide or polynucleotides encoding immunogenic polypeptides to be expressed may be inserted into any suitable region of the viral vector, for example into a deleted region.

Although two or more polynucleotides encoding immunogenic polypeptides may be linked as a fusion, the resulting protein may be expressed as a fusion protein, or it may be expressed as separate protein products, or it may be expressed as a fusion protein and then subsequently broken down into smaller subunits.

In one aspect the viral vector is suitably replication competent in the host organism to which it is to be delivered.

In a further aspect the viral vector is not affected by, or only minimally affected by the presence of an adjuvant. In one aspect any reduction in viral titer caused by the adjuvant is no more than 50%, such as no more than 40%, 30%, 20%, 15%, 10%, 5% and in a further aspect there is no reduction in titer at all.

### Adjuvant

Adjuvants are described in general, e.g. in Vaccine Design—the Subunit and Adjuvant Approach, Powell and Newman, Plenum Press, New York, 1995.

Suitable adjuvants include an aluminium salt such as aluminium hydroxide or aluminium phosphate, but may also be a salt of calcium, iron or zinc, or may be an insoluble suspension of acylated tyrosine, or acylated sugars, cationically or anionically derivatised polysaccharides, or polyphosphazenes.

In the formulation of the invention it is preferred that the adjuvant composition preferentially induces a Th1 response. However it will be understood that other responses, including other humoral responses, are not excluded.

It is known that certain vaccine adjuvants are particularly suited to the stimulation of either Th1 or Th2-type cytokine responses. Traditionally the best indicators of the Th1:Th2 balance of the immune response after a vaccination or infection includes direct measurement of the production of Th1 or Th2 cytokines by T lymphocytes in vitro after restimulation with antigen, and/or the measurement of the IgG1:IgG2a ratio of antigen specific antibody responses.

Thus, a Th1-type adjuvant is one which stimulates isolated T-cell populations to produce high levels of Th1-type cytokines in vivo (as measured in the serum) or ex vivo (cytokines that are measured when the cells are re-stimulated with antigen in vitro), and induces antigen specific immunoglobulin responses associated with Th1-type isotype.

Preferred Th1-type immunostimulants which may be formulated to produce adjuvants suitable for use in the present invention include and are not restricted to the following:

The Toll like receptor (TLR) 4 ligands, especially an agonist such as a lipid A derivative particularly monophosphoryl lipid A or more particularly 3 Deacylated monophoshoryl lipid A (3D-MPL).

3D-MPL is sold under the trademark MPL® by GlaxoSmithKline and primarily promotes CD4+ T cell responses characterized by the production of IFN-gamma (Th1 cells i.e. CD4 T helper cells with a type-1 phenotype). It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3D-MPL has a particle size such that it may be sterile-filtered through a 0.22 μm filter. Such preparations are described in International Patent Application No. WO94/21292. Synthetic derivatives of lipid A are known and thought to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3--[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Saponins are also preferred Th1 immunostimulants in accordance with the invention. Saponins are well known adjuvants and are taught in: Lacaille-Dubois, M and Wagner H. (1996. A review of the biological and pharmacological activities of saponins. Phytomedicine vol 2 pp 363-386). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711. One such system is known as an Iscorn and may contain one or more saponins.

The adjuvant of the present invention may in particular comprises a Toll like receptor (TLR) 4 ligand, especially 3D-MPL, in combination with a saponin.

Other suitable adjuvants include TLR 9 ligands (agonists). Thus another preferred immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon γ and have cytolytic activity) and macrophages (Wooldrige et al Vol 89 (no. 8), 1977). Other unmethylated CpG containing sequences not having this consensus sequence have also now been shown to be immunomodulatory.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).

Other TLR9 agonists of potential interest include immunostimulatory CpR motif containing oligonucleotides and YpG motif containing oligonucleotides (Idere).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra; Brazolot-Millan supra) or with other cationic carriers.

Combinations of immunostimulants are also preferred, in particular a combination of a monophosphoryl lipid A and a saponin derivative (WO 94/00153; WO 95/17210; WO 96/33739; WO 98/56414; WO 99/12565; WO 99/11241), more particularly the combination of QS21 and 3D-MPL as disclosed in WO 94/00153. Alternatively, a combination of CpG plus a saponin such as QS21 also forms a potent adjuvant for use in the present invention. Alternatively the saponin may be formulated in a liposome or in an Iscorn and combined with an immunostimulatory oligonucleotide.

Thus, suitable adjuvant systems include, for example, a combination of monophosphoryl lipid A, preferably 3D-MPL, together with an aluminium salt (eg as described in WO00/23105).

An enhanced system involves the combination of a monophosphoryl lipid A and a saponin derivative particularly the combination of QS21 and 3D-MPL as disclosed in WO 94/00153, or a less reactogenic composition where the QS21 is quenched in cholesterol containing liposomes (DQ) as disclosed in WO 96/33739. This combination may additionally comprise an immunostimulatory oligonucleotide.

Thus an example adjuvant comprises QS21 and/or MPL and/or CpG.

A particularly potent adjuvant formulation involving QS21, 3D-MPL & tocopherol in an oil in water emulsion is described in WO 95/17210 and is another preferred formulation for use in the invention.

Another preferred formulation comprises a CpG oligonucleotide alone or together with an aluminium salt.

In a further aspect of the present invention there is provided a method of manufacture of a vaccine formulation as herein described, wherein the method comprises admixing one or more first immunogenic polypeptides according to the invention with a suitable adjuvant.

Particularly preferred adjuvants for use in the formulations according to the invention are as follows:

i) 3D-MPL+QS21 in a liposome (see eg Adjuvant B below)

### ii) Alum+3D-MPL

iii) Alum+QS21 in a liposome+3D-MPL

### iv) Alum+CpG

v) 3D-MPL+QS21+oil in water emulsion

### vi) CpG

vii) 3D-MPL+QS21 (eg in a liposome)+CpG
viii) QS21+CpG.

Preferably, the adjuvant is presented in the form of a liposome, ISCOM or an oil-in-water emulsion. In one example embodiment of the invention the adjuvant comprises an oil-in-water emulsion. In another example embodiment of the invention the adjuvant comprises liposomes.

Suitably the adjuvant component does not contain any virus. Thus suitably, compositions for use according to the invention do not contain any virus other than the one or more more viral vectors comprising one or more heterologous polynucleotides encoding one or more second immunogenic polypeptides derived from a pathogen.

### Compositions, Dosage and Administration

In methods of the invention, the immunogenic polypeptide(s), the viral vector(s) and the adjuvant are administered concomitantly.

Typically the adjuvant will be co-formulated with an immunogenic polypeptide. Suitably the adjuvant will also be co-formulated with any other immunogenic polypeptide to be administered.

Thus in one embodiment of the invention there is provided a method of raising an immune response which comprises administering (i) one or more first immunogenic polypeptides co-formulated with an adjuvant; and (ii) one or more viral vectors comprising one or more heterologous polynucleotides encoding one or more second immunogenic polypeptides; wherein one or more first immunogenic polypeptides and adjuvant, and one or more viral vectors are administered concomitantly.

By “co-formulated” is meant that the first immunogenic polypeptide and the adjuvant are contained within the same composition eg a pharmaceutical composition.

Typically the viral vector is contained in a composition eg a pharmaceutical composition.

Alternatively, the one or more first immunogenic polypeptides, the one or more viral vectors and an adjuvant are co-formulated.

Thus, there are provided compositions according to the invention which comprise one or more immunogenic polypeptides, one or more viral vectors, and an adjuvant.

Compositions and methods according to the invention may involve use of more than one immunogenic polypeptide and/or more than one viral vector. Use of multiple antigens is especially advantageous in raising protective immune responses to certain pathogens, such as HIV, M. tuberculosis and Plasmodium sp. Compositions according to the invention may comprise more than one adjuvant.

Compositions and methods employed according to the invention may typically comprise a carrier eg an aqueous buffered carrier. Protective components such as sugars may be included.

Compositions should be administered in sufficient amounts to transduce the target cells and to provide sufficient levels of gene transfer and expression and to permit pathogen-specific immune responses to develop thereby to provide a prophylactic or therapeutic benefit without undue adverse or with medically acceptable physiological effects, which can be determined by those skilled in the medical arts. Conventional and pharmaceutically acceptable routes of administration include, but are not limited to, direct delivery to the retina and other intraocular delivery methods, direct delivery to the liver, inhalation, intranasal, intravenous, intramuscular, intratracheal, subcutaneous, intradermal, epidermal, rectal, oral and other parenteral routes of administration. Routes of administration may be combined, if desired, or adjusted depending upon the gene product or the condition. The route of administration primarily will depend on the nature of the condition being treated. Most suitably the route is intramuscular, intradermal or epidermal.

Preferred tissues to target are muscle, skin and mucous membranes. Skin and mucous membranes are the physiological sites where most infectious antigens are normally encountered.

When the first immunogenic polypeptide, adjuvant and viral vector are not co-formulated, the different formulations (eg polypeptide/adjuvant and viral vector formulations) may be administered by the same route of administration or by different routes of administration.

Dosages of compositions in the methods will depend primarily on factors such as the condition being treated, the age, weight and health of the subject, and may thus vary among subjects. For example, a therapeutically effective adult human or veterinary dosage is generally in the range of from about 100 μL to about 100 mL of a carrier containing concentrations of from about 1×10³ to about 1×10¹⁵ particles, such as 1×10⁶ to about 1×10¹⁵ particles, about 1×10¹¹ to 1×10¹³ particles, or about 1×10⁹ to 1×10¹² particles of virus together with around 1-1000 ug, or about 2-100 ug eg around 4-40 ug immunogenic polypeptide. For measles viral vectors a dose range of 1×10³ to 1×10⁶ particles may be used. Dosages will range depending upon the size of the animal and the route of administration. For example, a suitable human or veterinary dosage (for about an 80 kg animal) for intramuscular injection is in the range of about 1×10⁹ to about 5×10¹² virus particles and 4-40 ug protein per mL, for a single site. One of skill in the art may adjust these doses, depending on the route of administration, and the therapeutic or vaccinel application for which the composition is employed.

The amount of adjuvant will depend on the nature of the adjuvant and the immunogenic polypeptide, the condition being treated and the age, weight and health of the subject. Typically for human administration an amount of adjuvant of 1-100 ug eg 10-50 ug per dose may be suitable.

Suitably an adequate immune response is achieved by a single concomitant administration of the composition or compositions of the invention in methods of the invention. However if the immune response is further enhanced by administration of a further dose of first immunogenic polypeptide, adjuvant and viral vector on a second or subsequent occasion (for example after a month or two months) then such a protocol is embraced by the invention.

We have found that good pathogen-specific CD4+ and/or CD8+ T-cell responses may typically be raised after a single concomitant administration of the composition or compositions of the invention in methods of the invention. However we have found that good pathogen-specific antibody responses may require a second or further concomitant administration of the composition or compositions of the invention.

The components of the invention may be combined or formulated with any suitable pharmaceutical excipient such as water, buffers and the like.

In one aspect of the invention, co-formulation or co-administration of the composition as claimed provides an additive effect on, or synergistic increase in, the CD4 and/or CD8 responses obtained, for example as determined using the assay techniques disclosed herein.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims appended herein:

The following examples and data illustrate the invention, but are not limiting upon the invention.

### 1. Adjuvant Preparations

### 1.1 The Preparation of Oil in Water Emulsion Followed the Protocol as Set Forth in WO 95/17210.

The emulsion contains: 42.72 mg/ml squalene, 47.44 mg/ml tocopherol, 19.4 mg/ml Tween 80. The resulting oil droplets have a size of approximately 180 nm Tween 80 was dissolved in phosphate buffered saline (PBS) to give a 2% solution in the PBS. To provide 100 ml two fold concentrate, emulsion 5 g of DL alpha tocopherol and 5 ml of squalene were vortexed until mixed thoroughly. 90 ml of PBS/Tween solution was added and mixed thoroughly. The resulting emulsion was then passed through a syringe and finally microfluidised by using an M110S microfluidics machine. The resulting oil droplets have a size of approximately 180 nm

1.2 Preparation of Oil in Water Emulsion with QS21 and MPL

Sterile bulk emulsion was added to PBS to reach a final concentration of 500 μl of emulsion per ml (v/v). 3 D-MPL was then added. QS21 was then added Between each addition of component, the intermediate product was stirred for 5 minutes. Fifteen minutes later, the pH was checked and adjusted if necessary to 6.8+/−0.1 with NaOH or HCl. The final concentration of 3D-MPL and QS21 was 100 μg per ml for each.

### 1.3 Preparation of Liposomal MPL

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 10 mg/ml.

The final concentration of MPL is 2 mg/ml.

The liposomes have a size of approximately 100 nm and are referred to as SUV (for small unilamelar vesicles). The liposomes by themselves are stable over time and have no fusogenic capacity.

### 1.4 Preparation of Adjuvant B (“adj B”)

Sterile bulk of SUV was added to PBS. PBS composition was Na₂HPO₄: 9 mM; KH₂PO₄: 48 mM; NaCl: 100 mM pH 6.1. QS21 in aqueous solution was added to the SUV. The final concentration of 3D-MPL and QS21 was 100 μg per ml for each. This mixture may be referred as Adjuvant B. Between each addition of component, the intermediate product was stirred for 5 minutes. The pH was checked and adjusted if necessary to 6.1+/−0.1 with NaOH or HCl.

### 2. Preparation of HIV Antigens

2.1 p24-RT-Nef-P17 Protein (“F4”)

F4 was prepared as described in WO2006/013106 Example 1, codon-optimised method.

### 3. Preparation of Measles Viral Vectors

### 3.1 Rescue of MV1-F4 Virus

MV1-F4 virus was rescued using a helper cell line and amplified on Vero cells as described in Combredet C, Labrousse V, Mollet L, Lorin C, Delebecque F, Hurtrel B, McClure H, Feinberg M B, Brahic M, and Tangy F (2003) A molecularly cloned Schwarz strain of measles virus vaccine induces strong immune responses in macaques and transgenic mice. J Virol, 77, 11546-11554.).

4. Co-Administration and Co-Formulation Strategies Using Both F4/adjB and MV1-F4

The F4 protein has been shown to induce strong HIV-specific CD4 T cells in mice, rhesus monkeys and humans when administrated with the adjB adjuvant intra-muscularly.

In addition to the F4/adjB vaccine candidate, MV1-F4 constitutes another vaccine candidate using the F4 antigen.

The added value of using both F4/adjB and MV1-F4 in co-administration and co-formulation strategies on the quality and intensity of F4-specific T cell responses in a mouse model was assessed. Adjuvant B is also referred to as AS01B herein.

### Studies in Mice

### 4.1 Mice and Immunisations

FVB mice heterozygous for the hCD46 transgene (a kind gift from F. Grosveld, Erasmus University, Rotterdam, The Netherlands) were crossed with 129sv IFN-α/α R−/− mice which lack the type-I IFN receptor (a kind gift from M. Aguet, Swiss Institute for Experimental Cancer Research, Epalinges, Switzerland). The F1 progeny was screened by PCR, and the CD46+/− animals were crossed again with 129sv IFN-α/α R−/− mice. IFN-α/α R−/− CD46+/− were selected and used for immunization experiments. These mice are susceptible to MV infection. Mice were housed under specific pathogen-free conditions at the Pasteur Institute animal facility. Ten to 12-week-old female CD46+/− IFN-α/α R−/− (CD46/IFNAR) mice were inoculated intraperitoneally or intramuscularly with various doses of MV1-F4 and intramuscularly with 9 or 18 μg of recombinant F4 protein mixed in 100 μl adjB. Seven days after immunisation, mice were euthanized and splenocytes were collected. All experiments were approved and conducted in accordance with the guidelines of the Office of Laboratory Animal Care at Pasteur Institute.

### 4.2 Cell-Mediated Immune Response Analysis

Splenocytes from immunised mice were tested for their capacity to secrete IFN-γ upon specific stimulation by flow cytometry. Spleen cells were cultured for 6 h in 48-well plates (Costar) at concentration of 2.10⁶ cells/well in a volume of 0.4 ml complete medium (RPMI 1640/glutamax medium supplemented with 5% fetal calf serum, 50 mM 2-mercapto-ethanol, non essential amino acids, sodium pyruvate and antibiotics) either with or without pools of peptides covering the F4 sequence (1 μg/ml each peptide final concentration). Brefeldin A (10 μg/ml) was then added overnight. Cells were harvested, washed in phosphate-buffered saline containing 1% bovine serum albumin and 0,1% sodium azide (FACS buffer), incubated 10 min with Fc blocking Ab and surface stained in FACS buffer with anti-CD4-PE and anti-CD8-PerCP for 30 min at 4° C. in the dark. After washing the unbound antibody, cells were fixed and permeabilised for intracellular cytokine stain using the Cytofix/Cytoperm kit according to the manufacturer's instructions (BD). Cells were then incubated in a mix of anti IFNγ-APC/anti-IL2-FITC diluted in permwash buffer (BD) for 45 min in the dark. After washing with permwash buffer and FACS buffer, cells were finally fixed with 1% formaldehyde in PBS. Twenty thousand events in CD8 gate were acquired using a FACSCalibur flow cytometer (Becton Dickinson). Data were analysed using CELLQuest software (Becton Dickinson) and presented as % of CD4 or CD8 cells expressing IL-2 or IFNγ among CD4 or CD8 population. The following antibodies were used: Fluorescein isothiocyanate (FITC)-conjugated rat anti mouse IL2 mAb (clone JES6-5H4), Phycoerythrin (PE)-conjugated rat anti mouse CD4 mAb (clone RM4-5), Peridinin chlorophyll protein (PerCP) conjugated rat anti mouse CD813 mAb (clone 53-6.7), allophycocyanin (APC) conjugated rat anti mouse IFNγ (clone XMG1.2) and Fc blocking CD16/32 (clone 2.4G2), which were purchased from PharMingen.

4.3 Co-administration protocol

In order to assess the added value of immunizing with both F4/adjB and MV1-F4 at the same time on the F4-specific T cell responses, both candidates were co-administered at two different sites in CD46/IFNAR mice.

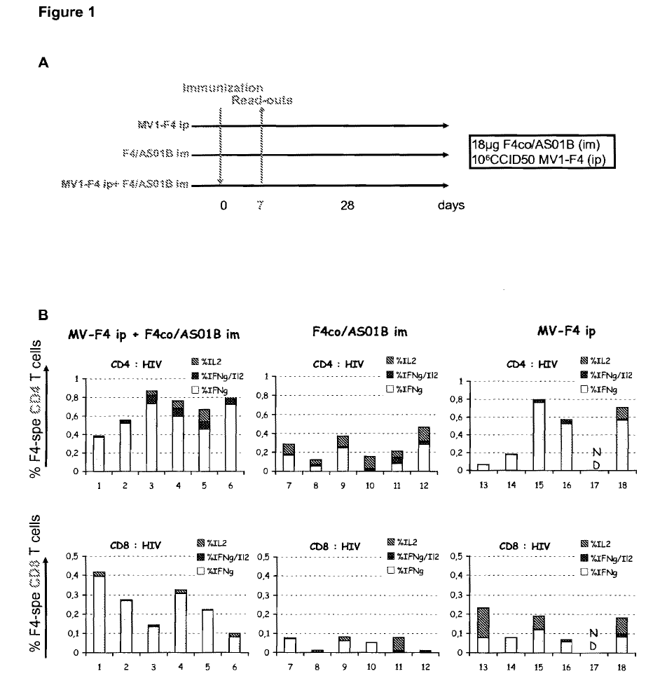

In a first set of experiments, mice received one injection of high doses of F4/adjB (18 μg, im) and MV1-F4 (10⁶CCID₅₀, ip) and the F4-specific T cell responses were analyzed 7 days post-immunization (FIG. 1A.).

Results show that F4/adjB alone induces mainly F4-specific CD4 T cells and MV1-F4 alone elicits both F4-specific CD4 and CD8 T cells. Interestingly, the intensity of both F4-specific CD4 and CD8 T cell responses is increased in mice receiving both candidates in co-administration (FIG. 1B).

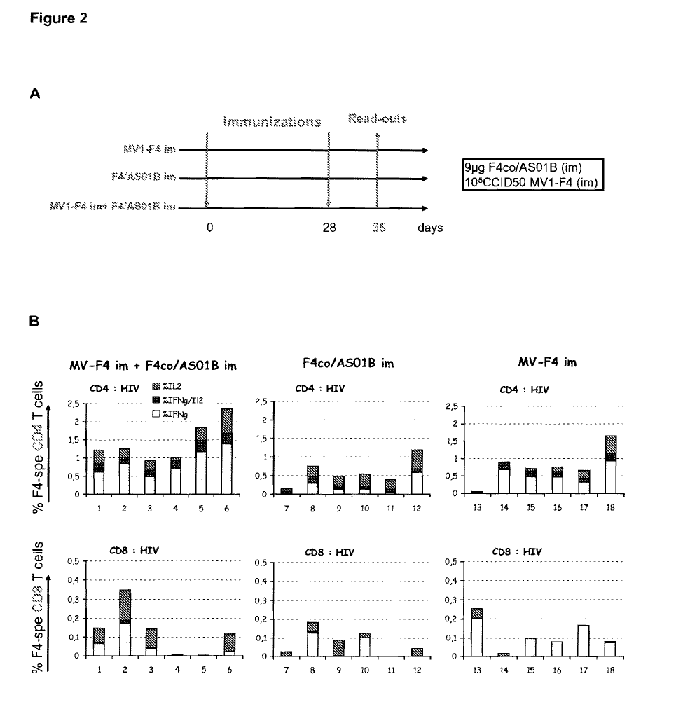

In a second set of experiments, mice received two injections at one month interval of lower doses of F4/adjB (9 μg) and MV1-F4 (10⁶CCID₅₀) and both candidates were injected intramuscularly at two different sites. The F4-specific T cell responses were analyzed 7 days post-second immunization (FIG. 2A).

Results show that the magnitude of F4-specific CD4 and CD8 T cell responses is higher in mice receiving the co-administration of F4/adjB and MV1-F4 than mice receiving each candidate alone.

All together, these results suggest that co-administrating both F4/adjB and MV1-F4 provides a synergistic effect on the magnitude of F4-specific CD4 and CD8 T cell responses. However, the profile of cytokine production of F4-specific T cells seems unchanged whether the candidates are injected alone or in co-administration.

### 4.4 Co-Formulation Protocol

MV1-F4 was mixed with the adjuvant adjB or medium and incubated for various periods of time at room temperature to assess the impact of adjuvant on the vector. MV1-F4 was then titrated on Vero cells and results show that a slight decrease of infectivity (around 0.5 Log) is observed when MV1-F4 is incubated with adjB as compared to the medium.

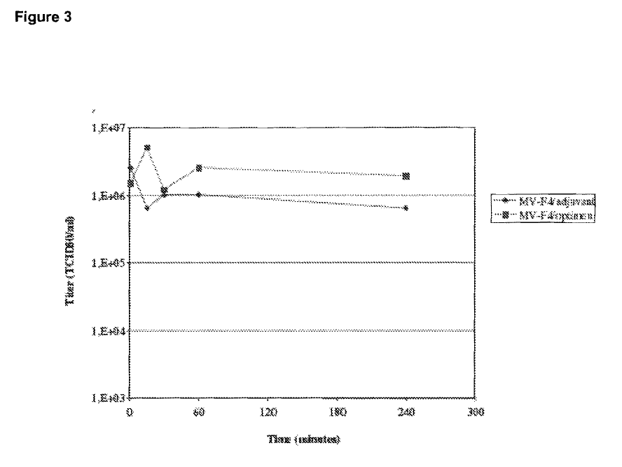

Results are shown in FIG. 3. MV1-F4 virus was incubated with adjB adjuvant or medium (OptiMEM) for the indicated time at room temperature. Then the viral titers were assessed on Vero cells by end-point serial dilution assay. The viral titers are expressed in TCID₅₀/mL.

### Studies in Monkeys

4.5 Immunogenicity of Vaccine Regimens in NHP

Cynomolgus macaques (N=10/group) were immunized twice at days 0, and 28 with the following vaccine regimen: (1) 10 μg F4co/AS01B (P), (2) 4.2 Log CCID₅₀ MV1-F4 (M) and (3) co-administration of both vaccine candidates (Co-ad). Immunogenicity of each vaccine regimen was monitored over time, up to 3 months post-last injection.

4.5.1 F4-specific CD4+ and CD8+ T Cell Responses Induced by the Various Vaccine Regimens.

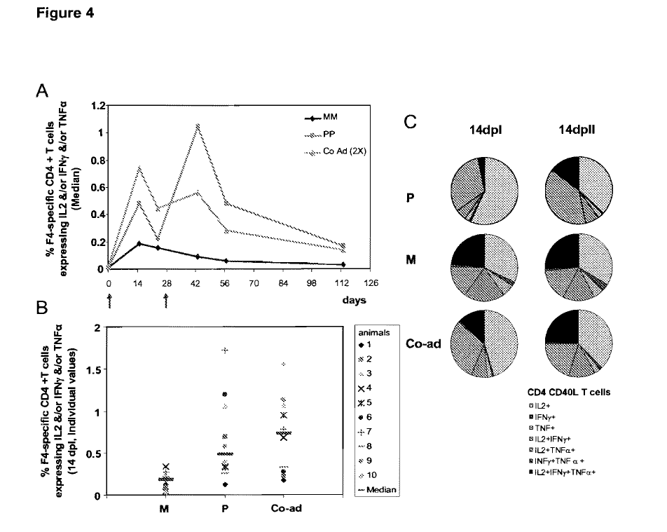

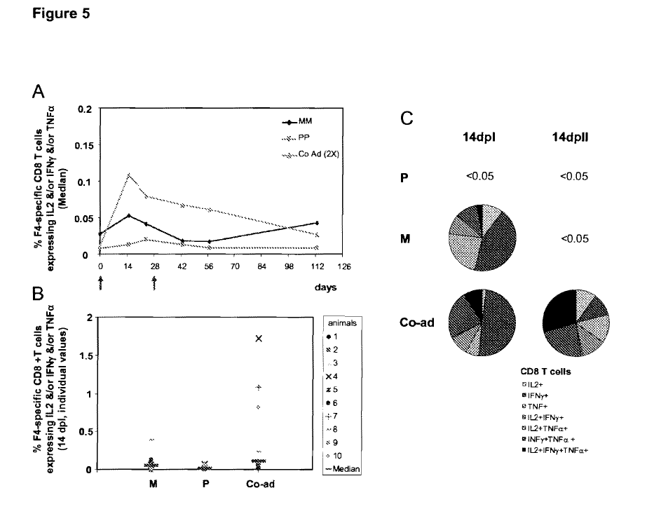

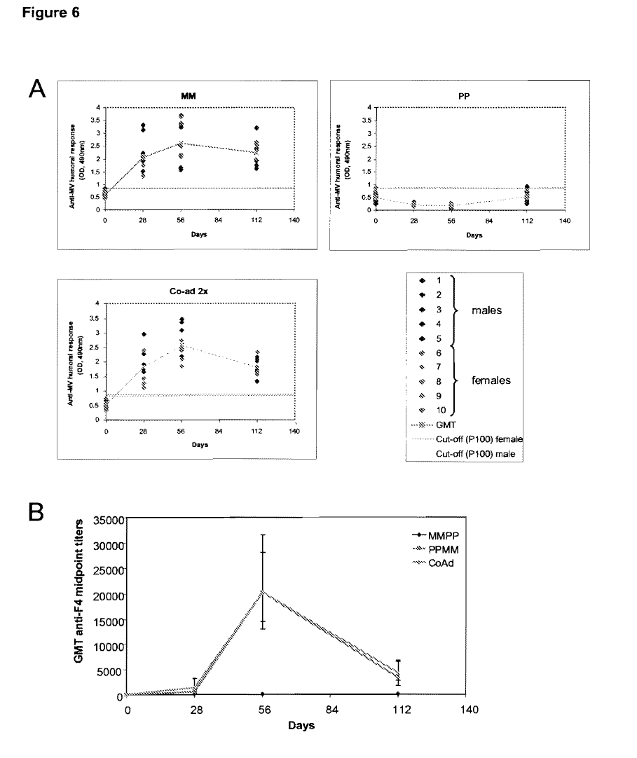

One injection of F4co/AS01B elicited a significant level of F4-specific CD4+ T cells, as detected in peripheral blood, (median value of 10 animals at 14 days post-I: 0.48% of total CD4+ T cells) and the second injection induced a very high frequency of specific CD4+ T cells with a median value of 1.04% at 14 days post-II (see FIG. 4A). All animals were responders against the F4co antigen, from the first dose (cut-off=0.05%), (see FIG. 4B). Interestingly, the specific CD4+ T cell response was still detectable three months post-last immunization (median value of 10 animals: 0.16%). F4-specific CD8+ T cells were not observed with this vaccine regimen (see FIGS. 5A and 5B).

The first injection of 4.2 Log CCID₅₀ MV1-F4 induced a detectable F4-specific CD4+ T cell response, but the intensity (median value of 10 animals at 14 days post-I: 0.18% of total CD4+ cells) was lower than the intensity of the F4co/AS01B-mediated F4-specific CD4+ T cell response (see FIG. 4A). At 14 days post-first immunization, only 8 out of 10 animals raised a F4-specific CD4+ T cell response but the two non-responder macaques raised a F4-specific CD4+ T cell response which was detectable at 28 days post-I. As a result, all animals developed F4-specific CD4+ T cell responses with the kinetics varying between individuals. F4-specific CD8+ T cells were detected in 4 out of 10 animals (cut-off=0.06%) (see FIG. 5B), 14 days after the first injection and an additional animal raised a specific CD8+ T cell response at 28 days post-I. A second injection of 4.2 Log CCID₅₀ MV1-F4 did not increase the frequencies of F4-specific CD4+ or CD8+ T cell responses.

Monkeys immunized with the co-administration regimen developed a F4-specific CD4+ T cell response comparable to the one raised in animals immunized with the F4co/AS01B vaccine candidate at 14 days post-I and post-II immunizations (median value of 10 animals at 14 days post-I: 0.73% and at 14 days post-II: 0.55% of total CD4+ T cells) (see FIG. 4A). All animals were responders against the F4co antigen, from the first dose (see FIG. 4B) and the specific CD4+ T cell response was still detectable three months post-last immunization (median value of 10 animals: 0.13%) (FIG. 4A). Interestingly, F4-specific CD8+ T cells were observed in 7 out of 10 animals at 14 days post-one injection and the frequency was high in three of these responders (% F4-specific CD8+ T cells=0.82, 1 and 1.7) (see FIG. 5B). The frequencies of F4-specific CD8+ T cells detected in these three macaques immunized with the co-administration regimen were higher than the ones observed in monkeys which were immunized with MV1-F4 alone (median value in the “M” group at 14 days post-I: 0.052% and median value in the “Co-ad” group at 14 days post-I: 0.1%) (see FIG. 5B). The second immunization with the co-administration regimen did not increase the intensity of F4-specific CD8+ T cell responses.

To summarize, monkeys which were immunized with the co-administration regimen raised a potent F4-specific CD4+ T cell response comparable to the F4co/AS01B-mediated CD4+ T cell response, in terms of intensity, and this specific response was still detectable up to three months post-second immunization. Interestingly, 7 out of 10 animals raised also a F4-specific CD8+ T cell response and a high frequency of F4-specific CD8+ T cells was observed in 3 of these animals. The second immunization with the co-administration protocol did not increase the number of responders or the level of F4-specific CD8+ T cell responses. As a result, the co-administration regimen favors the induction of both CD4+ and CD8+ T cells.

### 4.5.2 Cytokine Co-Expression Profile

The cytokine co-expression profile of F4-specific CD4+ and CD8+ T cells was assessed at 14 days post-one and post-two immunizations for the three vaccine regimens.

After the first dose of F4co/AS01B, the F4-specific CD4+ T-cells secreted mainly IL-2 alone or in combination with TNF-α (see FIG. 4C). The second dose of F4co/AS01B tends to increase the proportion of polyfunctional CD4+ T cells producing at least two or three cytokines. Interestingly, the proportion of F4-specific CD4+ T cells producing at least three cytokines tends to be higher in animals which received the co-administration protocol (Mean of 10 animals: 13% at 14 days post-1 and 24% at 14 days post-II) compared to the proportion observed in animals which received F4co/AS01B alone (Mean of 10 animals: 3% at 14 days post-1 and 14% at 14 days post-II) (see FIG. 4C).

F4-specific CD8+ T cells induced by MV1-F4 alone or the co-administration protocol produced mainly IFNγ alone or in combination with TNF-α. The second dose of the co-administration protocol tends to increase the proportion of polyfunctional F4-specific CD8+ T cells although no impact on the intensity of the global F4-specific CD8+ T cell response was observed (see FIGS. 5A and 5C).

Overall, the second immunization of each vaccine regimen tends to increase the proportion of F4-specific T cells secreting at least 3 cytokines. Interestingly, the added value of the co-administration regimen over F4co/AS01B alone on the proportion of polyfunctional F4-specific CD4+ T cells is observed from the first immunization.

### 4.5.3 Humoral Responses Induced by the Various Vaccine Regimens.

All animals which received the MV1-F4 candidate alone or the co-administration regimen developed an anti-MV humoral response after the first dose, demonstrating the intake of the MV1-F4 vaccine candidate. The second dose increased the level of anti-MV antibodies in both groups. The intensities of anti-MV humoral responses induced by MV1-F4 alone or the co-administration regimen were similar (see FIG. 6A).

Regarding the F4-specific humoral response, only animals immunized with two doses of the F4co/AS01B vaccine candidate and the co-administration regimen raised significant levels of anti-F4co antibodies. At 28 days post-two immunization, the anti-F4co mid-point titers were similar in both groups (geometric mean of 10 animals=20384 in the “P” group and =20365 in the “co-ad” group) (see FIG. 6B). The anti-F4co humoral response was low to undetectable in the animals which received 4.2 Log CCID₅₀ MV1-F4.

In conclusion, the pre-clinical data described herein demonstrates the immunogenicity of the co-administration regimen combining the F4co/AS01B and the MV1-F4 candidate vaccines in non-human primates. The co-administration regimen induced a very high specific CD4+ T cell response with a polyfunctional profile of cytokine secretion and a good persistence. The intensity of F4-specific CD4+ T cell responses induced by the co-administration protocol was comparable to the one induced by F4co/AS01B alone, but the proportion of polyfunctional F4-specific CD4+ T cells tends to be higher with the co-administration regimen. Interestingly, the co-administration regimen triggers the induction of F4-specific CD8+ T cells, in a significant proportion of animals, in addition to the F4-specific CD4+ T cell response.

1. An immunogenic composition comprising (i) one or more first immunogenic polypeptides from a pathogen; (ii) one or more viral vectors comprising one or more heterologous polynucleotides encoding one or more second immunogenic polypeptides from the pathogen; and (iii) an adjuvant.

2. The immunogenic composition of claim 1, wherein the one or more first immunogenic polypeptides from the pathogen are co-formulated with an adjuvant.

3.-8. (canceled)

9. The immunogenic composition of claim 1, wherein one or more of said one or more first immunogenic polypeptides is substantially the same as or contains at least one antigen in common with one or more of said one or more second immunogenic polypeptides.

10. (canceled)

11. The immunogenic composition of claim 1, wherein the one or more first immunogenic polypeptides comprises at least one of a T cell epitope and a B cell epitope.

12.-13. (canceled)

14. The immunogenic composition of claim 1, wherein none of the one or more of said one or more first immunogenic polypeptides is substantially the same as or contains any antigen in common with one or more of said one or more second immunogenic polypeptides.

15. The immunogenic composition of claim 1, wherein the pathogen is HIV.

16. The immunogenic composition of claim 15, wherein the first and/or second immunogenic polypeptides contain HIV antigens selected from the group of: Env, Nef, Gag, and Pol and immunogenic fragments thereof.

17. The immunogenic composition of claim 16, wherein a first immunogenic polypeptide is p24-RT-Nef-p 17.

18. The immunogenic composition of claim 16, wherein a second immunogenic polypeptide is Gag-RT-Nef.

19. The immunogenic composition of claim 1, wherein the pathogen is Plasmodium falciparum and/or Plasmodium vivax.

20. The immunogenic composition of claim 1, wherein the first and/or second immunogenic polypeptides contain antigens derived from Plasmodium falciparum and/or Plasmodium vivax which are selected from the group of: circumsporozoite (CS) protein, MSP-1, MSP-3, AMA-1, LSA-1, LSA-3 and immunogenic derivatives thereof or immunogenic fragments thereof.

21. The immunogenic composition of claim 19, wherein a first and or second immunogenic polypeptide is a hybrid protein RTS.

22.-23. (canceled)

24. The immunogenic composition of claim 1, wherein the pathogen is Mycobacterium tuberculosis.

25. The immunogenic composition of claim 1, wherein the adjuvant comprises a preferential stimulator of Th1 responses.

26. The immunogenic composition of claim 25, wherein the adjuvant comprises at least one of QS21, 3D-MPL, a CpG oligonucleotide, an oil-in-water emulsion, and a liposome.

27.-32. (canceled)

33. A kit comprising (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant.

34.-35. (canceled)

36. The immunogenic composition of claim 1, wherein the viral vector is an attenuated measles viral vector.

37.-38. (canceled)

39. A method of raising an immune response in a mammal comprising administering to the mammal (i) one or more first immunogenic polypeptides derived from a pathogen; (ii) one or more viral vectors comprising one or more heterologous polynucleotides encoding one or more second immunogenic polypeptides derived from said pathogen; and (iii) an adjuvant; wherein the one or more first immunogenic polypeptides, the one or more viral vectors and the adjuvant are administered concomitantly; and optionally repeating the administering one or more times.

40. The method of claim 39, wherein the administering stimulates the production of pathogen-specific CD4+ and/or CD8+ T-cells and/or antibodies in the mammal.

41. The method of claim 39, wherein the administering is not preceded by administering any priming dose of immunogenic polypeptide or polynucleotide encoding immunogenic polypeptide.
